(19) United States
(12) Patent Application Publication
(10) Pub. No.: US 2025/0034516 A1
(43) Pub. Date: Jan. 30, 2025

(54) METHODS FOR CELL CULTURING

(71) Applicant: Orbillion Bio, Inc., San Francisco, CA (US)

(72) Inventors: Azadeh Mostafavi, San Francisco, CA (US); Laura Daley, San Francisco (CA); Gabriel Levesque-Tremblay, San Francisco, CA (US)

(73) Assignee: Orbillion Bio, Inc., San Francisco, CA (US)

(21) Appl. No.: 18/715,599

(22) PCT Filed: Jan. 24, 2023
(86) PCT No.: PCT/US2023/061128

§ 371 Date: May 31, 2024

Related U.S. Application Data
(60) Provisional application No. 63/302,842, filed on Jan. 25, 2022.

(51) Int. Cl. C12N 5/00; A23J 3/06; A23J 3/22; C12M 1/12; C12N 5/077

(52) U.S. Cl. CPC C12N 5/0068; A23J 3/06; A23J 3/227; C12M 25/14; C12N 5/0658

(57) ABSTRACT

Described herein is a method of acquiring a scaffold for a cultivated meat product comprising: (a) forming hydrogel fibers; (b) crosslinking the hydrogel fibers to obtain crosslinked hydrogel fibers; and (c) lyophilizing the crosslinked hydrogel fibers to obtain crosslinked lyophilized hydrogel fibers.

### CROSS-REFERENCE

This application claims the benefit of U.S. Provisional Application Ser. No. 63/302,842 filed on Jan. 25, 2022, the entirety of which is hereby incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Current methods for global meat production require substantial amounts of land, food, and other resources to raise traditional domesticated animals for consumption. These methods can be expensive, can generate pollution, and are not sustainable.

### SUMMARY

Accordingly, there remains a need for methods for meat production. Cultivated meat or cellular agriculture provides an alternative to the traditional meat production and can include the harvesting and scale-up of species-specific cells grown in vitro, thereby alleviating the number and cost of animals raised on rapidly declining land dedicated to animal husbandry to sustainably feed the growing population around the world.

Described herein, in some aspects, is a method of acquiring a scaffold for a cultivated meat product comprising: forming hydrogel fibers; crosslinking the hydrogel fibers to obtain crosslinked hydrogel fibers; and lyophilizing the crosslinked hydrogel fibers to obtain crosslinked lyophilized hydrogel fibers. In some embodiments, the method further comprises culturing cells on the crosslinked lyophilized hydrogel fibers. In some embodiments, the hydrogel fibers comprise: agarose, alginate, amino acid, cellulose, cellulose derivatives, chitosan, dextran, collagen, ethylene polyoxide, ethylene polyoxide copolymers, fibrin, gelatin, gelatin derivatives, hyaluronate, hyaluronan, hyaluronic acid methacrylate (HA-MA), hydroxyethyl methacrylate, lactic acid polymers, lipids, Matrigel™, natural polymers, Pluronic F-127, polyethylene glycol, polylactide-co-glycolide, polyacrylic acids, polyacrylic acids derivatives, polyvinyl alcohol, polyphosphazene, poloxamer, polysaccharides, proteins, peptides, poly-isopropyl-n-polyacrylamide, polyethylene glycol diacrylate (PEG-DA), polydimethylsiloxane, polyacrylamide, or any combination thereof. In some embodiments, the hydrogel fibers comprise: agarose, alginate, amino acid, cellulose, cellulose derivatives, chitosan, dextran, soy protein, pea protein, whey protein, starch, starch derivatives, insect derived proteins, fungi, collagen, fibrin, gelatin, gelatin derivatives, hyaluronate, hyaluronan, hyaluronic acid methacrylate (HA-MA), lipids, polyethylene glycol, polylactide-co-glycolide, polysaccharides, proteins, crosslinking precursor component(s) or any combination thereof. In some embodiments, the hydrogel fibers comprise gelatin. In some embodiments, the hydrogel fibers are formed from a concentration of about 1% to about 90% (w/v) hydrogel in an aqueous solution. In some embodiments, the hydrogel fibers are formed from a concentration of about 5% to about 30% (w/v) hydrogel in an aqueous solution. In some embodiments, the hydrogel fibers are formed from a concentration of about 5%, 6%, 7%, 8%, 9%, 10%, or 12% (w/v) hydrogel in an aqueous solution. In some embodiments, the hydrogel fibers are formed from a concentration of about 8% (w/v) hydrogel in an aqueous solution. In some embodiments, the hydrogel fibers are formed at about 4° C. to about 50° C. In some embodiments, the hydrogel fibers are formed at about 37° C. In some embodiments, forming the hydrogel fibers comprises filling an assembly fitted with a mesh with micro-sized apertures with liquid hydrogel, allowing the liquid hydrogel to cool and solidify to obtain solid hydrogel, and extruding the solid hydrogel through the fitted assembly mesh to obtain hydrogel fibers. In some embodiments, the assembly comprises an extruder. In some embodiments, the assembly comprises an assembly chamber pressure. In some embodiments, the assembly chamber pressure is from about 5 millitorr to about 4000 millitorr. In some embodiments, forming the hydrogel fibers comprises extrusion. In some embodiments, crosslinking the hydrogel fibers to obtain crosslinked hydrogel fibers comprises: suspending the hydrogel fibers in an aqueous solution comprising at least one crosslinking reagent to obtain suspended hydrogel fibers; and maintaining the suspended hydrogel fibers for at least 6 hours at a temperature from about 0° C. to about 10° C. to obtain crosslinked hydrogel fibers. In some embodiments, crosslinking the hydrogel fibers to obtain crosslinked hydrogel fibers comprises suspending the hydrogel fibers in an aqueous solution comprising at least one crosslinking reagent selected from a group consisting of: homobifunctional crosslinking reagents, heterobifunctional crosslinking reagents, phenolic crosslinking reagents, alginic acid, di- or polycarboxylic crosslinkers, oxidized polysaccharides, condensation involving cystamine/cysteine, photoreactive crosslinking reagents, disuccinimidyl suberate (DSS), disuccinimidyl tartrate (DST), dithiobis succinimidyl propionate (DSP), sulfhydryl-to-sulfhydryl crosslinkers, bismaleimidoethane (BMOE), dithiobismaleimidoethane (DTME), m-Maleimidobenzoyl-N-hydroxysuccinimide ester (MDS), N-γ-Maleimidobutyryloxysuccinimide ester (GMBS), N-(ε-Maleimidocaproyloxy) succinimide ester (EMCS), N-(ε-Maleimidocaproyloxy) sulfo succinimide ester (sulfo-EMCS), aryl azides, N-((2-pyridyldithio)ethyl)-4-azidosalicylamide, diazirines, transglutaminase, genipin, N-hydroxysuccinimide (NHS) ester, 1-Ethyl-3-[3-dimethylaminopropyl]carbodiimide hydrochloride (EDC or EDAC), hexamethylenetetramine, glutaraldehyde, epoxy compounds, isocyanates, or any combination thereof. In some embodiments, crosslinking the hydrogel fibers to obtain crosslinked hydrogel fibers comprises suspending the hydrogel fibers in an aqueous solution comprising at least one crosslinking agent selected from N-hydroxysuccinimide (NHS) ester, 1-Ethyl-3-[3-dimethylaminopropyl]carbodiimide hydrochloride (EDC or EDAC), genipin, transglutaminase, or any combination thereof. In some embodiments, crosslinking the hydrogel fibers to obtain crosslinked hydrogel fibers comprises shaker cultivating the suspended hydrogel fibers for 6 hours to 24 hours at a temperature from about 0° C. to about 37° C. In some embodiments, crosslinking the hydrogel fibers to obtain crosslinked hydrogel fibers comprises shaker cultivating the suspended hydrogel fibers for no more than 12 hours at a temperature from about 0° C. to about 37° C. In some embodiments, crosslinking the hydrogel fibers to obtain crosslinked hydrogel fibers comprises shaker cultivating the suspended hydrogel fibers from about 50 revolutions per minute to about 500 revolutions per minute. In some embodiments, crosslinking the hydrogel fibers to obtain crosslinked hydrogel fibers comprises shaker cultivating the suspended hydrogel fibers for 6 hours to 24 hours at a temperature from about 34° C. to about 40° C. In some embodiments, crosslinking the hydrogel fibers to obtain crosslinked hydrogel fibers comprises shaker cultivating the suspended hydrogel fibers for no more than 16 hours at a temperature from about 34° C. to about 37° C. In some embodiments, crosslinking the hydrogel fibers comprises a photo-crosslinking reaction. In some embodiments, the photo-crosslinking reaction comprises exposure to ultraviolet light. In some embodiments, the ultraviolet light comprises a wavelength of about 200 nanometers to about 600 nanometers. In some embodiments, the ultraviolet light comprises a wavelength of about 300 nanometers to about 400 nanometers. In some embodiments, lyophilizing the crosslinked hydrogel fibers to obtain crosslinked lyophilized hydrogel fibers comprises: freezing the crosslinked hydrogel fibers at a first temperature sufficient to transform the water in crosslinked hydrogel fibers from liquid state to solid state; and drying the crosslinked hydrogel fibers at a second temperature sufficient to remove the water by sublimation from the crosslinked hydrogel fibers. In some embodiments, the method further comprises washing the crosslinked hydrogel fibers. In some embodiments, the first temperature is from about −100° C. to about −10° C. In some embodiments, the second temperature is from about −50° C. to about 50° C. In some embodiments, the crosslinked hydrogel fibers are dried in a vacuum chamber at pressure from about 5 millitorr to about 4000 millitorr. In some embodiments, the lyophilized crosslinked hydrogel fibers are sterilized to obtain sterilized lyophilized crosslinked hydrogel fibers, wherein sterilization comprises: heating or irradiating the lyophilized crosslinked hydrogel fibers; and/or immersing the lyophilized crosslinked hydrogel fibers in alcohol-based soaking solution. In some embodiments, the heating comprises heating the lyophilized crosslinked hydrogel fibers with a temperate between about 37° C. to about 121° C. In some embodiments, the irradiating comprises contacting the lyophilized crosslinked hydrogel fibers with UV radiation. In some embodiments, the hydrogel fibers are characterized by a flexible dissolution rate from about 3 minutes to more than 100 days. In some embodiments, the hydrogel fibers are characterized by controllable gelation time from about 5 seconds to about 12 minutes. In some embodiments, the hydrogel fibers possess an average width from about 40 micrometers to about 1000 micrometers. In some embodiments, the hydrogel fibers possess an average width from about 40 micrometers to about 250 micrometers. In some embodiments, the hydrogel fibers possess an average length of from about 150 micrometers to about 12 centimeters. In some embodiments, the hydrogel fibers possess an average density from about 19 mole per cubic meter to about 56 mole per cubic meter. In some embodiments, the hydrogel fibers comprise low-rigidity elasticity from about 2 kilopascals to about 30 kilopascals. In some embodiments, the hydrogel fibers are water-stable. In some embodiments, the hydrogel fibers comprise a porous surface wherein an average pore size opening possesses a width from about 2 micrometers to about 500 micrometers. In some embodiments, the hydrogel fibers are bio-compatible. In some embodiments, the hydrogel fibers comprise a thermoreversible hydrogel that is not a liquid at room temperature. In some embodiments, the hydrogel fibers comprise a thermoreversible hydrogel with a gelation temperature (Tgel) from about 10° C. to about 40° C. In some embodiments, the method further comprises contacting the scaffold with cells or cell precursors from a non-human animal source. In some embodiments, the cells or cell precursors from a non-human animal source comprise cells from a tissue biopsy, an immortalized cell line, blood, stem cells, precursor cells, embryonic cells, bone marrow, or any combination thereof. In some embodiments, the method includes screening cells or cultured fibers for metabolic activity. In some embodiments, the method includes expanding the cells or cell precursors for 12 hours to 10 days to obtain cell cultured fibers. In some embodiments, the method includes expanding the cells or cell precursors for 12 hours to 10 days to obtain cell cultured fibers in suspension culture. In some embodiments, the method comprises harvesting the cell cultured fibers when the average width of the cell cultured fibers is from about 40 micrometers to about 2000 micrometers. In some embodiments, the cells comprise a single cell type. In some embodiments, the cells comprise a mixture of two or more cell types. In some embodiments, the single cell type or the two or more cell types are selected from muscle cells or muscle cell precursors, endothelial cells or endothelial cell precursors, adipose cells or adipose cell precursors, connective tissue cells of connective tissue cell precursors, or a combination thereof. In some embodiments, the cell cultured fibers further comprise embryonic stem cells, induced pluripotent stem cells, satellite cells, mesenchymal stem cells, and/or hematopoietic stem cells. In some embodiments, the non-human animal is selected from the group consisting of: a cow, a pig, a chicken, a fish, a sheep, a bison, a duck, a goose, an elk, a deer, a Berkshire pig, a Kurobuta pig, an Iberian pig, an ostrich, and combinations thereof. In some embodiments, the cell cultured fibers are cultured in a heterologous extracellular matrix.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates a method of making the hydrogel fibers described herein.

FIG. 2 illustrates a method of lyophilizing the hydrogel fibers described herein.

FIG. 3 illustrates a method of culturing cells on the hydrogel fibers described herein.

FIG. 4 depicts cells grown on hydrogel fibers using the methods described herein.

FIG. 5 illustrates that cells grown on hydrogel fibers can be admixed to form complex tissue compositions comprising two or more types of cells.

FIG. 6 illustrates proliferation of the fibers across seven days cultured by the method described herein.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Typically, two-dimensional (2D) cell cultures are used to understand the formation of tissue and organs, as well as diseases in vitro. However, 2D cell culture techniques do not directly replicate the mechanical and biochemical signals present in the body. In 2D techniques, cell-to-surface interactions prevail, rather than cell-to-cell and cell- to extracellular matrix (ECM) interactions that form the basis for normal cell function. Since awareness of the relevance of the cellular microenvironment has increased, three-dimensional (3D) cell culture is gaining popularity. 3D cell cultures facilitate the production of homotypic or heterotypic cell cultures in a spatially relevant manner that mimics the natural microenvironment.

Described herein, in some aspects, is a method for acquiring a scaffold for a cultivated meat product. In some embodiments, the method comprises: forming hydrogel fibers; crosslinking the hydrogel fibers to obtain crosslinked hydrogel fibers; and lyophilizing the crosslinked hydrogel fibers to obtain crosslinked lyophilized hydrogel fibers. In some embodiments, the crosslinking of the hydrogel comprises: suspending the hydrogel fibers in an aqueous solution comprising at least one crosslinking reagent to obtain suspended hydrogel fibers; or maintaining the suspended hydrogel fibers for at least 6 hours at a temperature from about 0° C. to about 37° C. to obtain crosslinked hydrogel fibers. In some embodiments, the crosslinked hydrogel fibers can be washed with water or ethanol to remove residue stemmed from the crosslinking reaction. In some embodiments, the lyophilizing of the hydrogel fibers comprises: freezing the crosslinked hydrogel fibers at a first temperature sufficient to transform the water in crosslinked hydrogel fibers from liquid state to solid state; or drying the crosslinked hydrogel fibers at a second temperature sufficient to remove the water by sublimation from the crosslinked hydrogel fibers. In some embodiments, the method comprises sterilizing the crosslinked hydrogel fibers. In some embodiments, the sterilizing of the crosslinked hydrogel fibers comprises: heating or irradiating the lyophilized crosslinked hydrogel fibers or immersing the lyophilized crosslinked hydrogel fibers in alcohol-based soaking solution.

### Methods

Described herein, in some aspects, is a method for acquiring a scaffold for a cultivated meat product. In some embodiments, the scaffold comprises hydrogel fibers. In some embodiments, the method comprises: forming hydrogel fibers; crosslinking the hydrogel fibers to obtain crosslinked hydrogel fibers; and lyophilizing the crosslinked hydrogel fibers to obtain crosslinked lyophilized hydrogel fibers. In some embodiments, the method comprises forming and crosslinking the hydrogel fibers and subsequently lyophilizing the crosslinked hydrogel fibers to obtain crosslinked lyophilized hydrogel fibers. In some embodiments, the lyophilized hydrogel fibers can be rehydrated. For example, the lyophilized hydrogel fibers can be rehydrated before being used for culturing of seeding of a cell described herein. In some embodiments, the crosslinked hydrogel fibers can be directly used for culturing or seeding of a cell described herein without lyophilizing the hydrogel fibers. In some embodiments, a cell cultured or seeded on the lyophilized hydrogel fibers (e.g., the hydrogel fibers that are rehydrated before or during the culturing or seeding of the cell) exhibits similar cellular function or viability as a comparable cell cultured or seeded on hydrogel fibers that have not been lyophilized.

In some embodiments, the hydrogel fibers comprise a hydrogel derived from agarose, alginate, amino acid, cellulose, cellulose derivatives, mycelium, bacterial nanocellulose, Pectin (carbohydrate-galacturonic acid complex), chitosan, soy protein, pea protein, whey protein, starch, starch derivatives, insect derived proteins, fungi, dextran, collagen, ethylene polyoxide, ethylene polyoxide copolymers, fibrin, gelatin, gelatin derivatives, hyaluronate, hyaluronan, hyaluronic acid methacrylate (HA-MA), hydroxyethyl methacrylate, lactic acid polymers, lipids, Matrigel™, natural polymers, Pluronic F-127, polyethylene glycol, polylactide-co-glycolide, polyacrylic acids, polyacrylic acids derivatives, polyvinyl alcohol, polyphosphazene, poloxamer, polysaccharides, proteins, peptides, poly-isopropyl-n-polyacrylamide, polyethylene glycol diacrylate (PEG-DA), polydimethylsiloxane, polyacrylamide, or any combination thereof or RGD modification or any modification to any above material than can help them to have better cell attachment.

In some embodiments, the hydrogel fibers comprise a percentage of hydrogel (w/v) formed in an aqueous solution. In some embodiments, the hydrogel fibers are formed from a concentration of about 1% to about 90% hydrogel in an aqueous solution. In some embodiments, the hydrogel fibers are formed from a concentration of about 0.1% hydrogel in an aqueous solution to about 90% hydrogel in an aqueous solution. In some embodiments, the hydrogel fibers are formed from a concentration of about 0.1% hydrogel in an aqueous solution to about 0.5% hydrogel in an aqueous solution, about 0.1% hydrogel in an aqueous solution to about 1% hydrogel in an aqueous solution, about 0.1% hydrogel in an aqueous solution to about 2% hydrogel in an aqueous solution, about 0.1% hydrogel in an aqueous solution to about 5% hydrogel in an aqueous solution, about 0.1% hydrogel in an aqueous solution to about 6% hydrogel in an aqueous solution, about 0.1% hydrogel in an aqueous solution to about 7% hydrogel in an aqueous solution, about 0.1% hydrogel in an aqueous solution to about 8% hydrogel in an aqueous solution, about 0.1% hydrogel in an aqueous solution to about 9% hydrogel in an aqueous solution, about 0.1% hydrogel in an aqueous solution to about 10% hydrogel in an aqueous solution, about 0.1% hydrogel in an aqueous solution to about 50% hydrogel in an aqueous solution, about 0.1% hydrogel in an aqueous solution to about 90% hydrogel in an aqueous solution, about 0.5% hydrogel in an aqueous solution to about 1% hydrogel in an aqueous solution, about 0.5% hydrogel in an aqueous solution to about 2% hydrogel in an aqueous solution, about 0.5% hydrogel in an aqueous solution to about 5% hydrogel in an aqueous solution, about 0.5% hydrogel in an aqueous solution to about 6% hydrogel in an aqueous solution, about 0.5% hydrogel in an aqueous solution to about 7% hydrogel in an aqueous solution, about 0.5% hydrogel in an aqueous solution to about 8% hydrogel in an aqueous solution, about 0.5% hydrogel in an aqueous solution to about 9% hydrogel in an aqueous solution, about 0.5% hydrogel in an aqueous solution to about 10% hydrogel in an aqueous solution, about 0.5% hydrogel in an aqueous solution to about 50% hydrogel in an aqueous solution, about 0.5% hydrogel in an aqueous solution to about 90% hydrogel in an aqueous solution, about 1% hydrogel in an aqueous solution to about 2% hydrogel in an aqueous solution, about 1% hydrogel in an aqueous solution to about 5% hydrogel in an aqueous solution, about 1% hydrogel in an aqueous solution to about 6% hydrogel in an aqueous solution, about 1% hydrogel in an aqueous solution to about 7% hydrogel in an aqueous solution, about 1% hydrogel in an aqueous solution to about 8% hydrogel in an aqueous solution, about 1% hydrogel in an aqueous solution to about 9% hydrogel in an aqueous solution, about 1% hydrogel in an aqueous solution to about 10% hydrogel in an aqueous solution, about 1% hydrogel in an aqueous solution to about 50% hydrogel in an aqueous solution, about 1% hydrogel in an aqueous solution to about 90% hydrogel in an aqueous solution, about 2% hydrogel in an aqueous solution to about 5% hydrogel in an aqueous solution, about 2% hydrogel in an aqueous solution to about 6% hydrogel in an aqueous solution, about 2% hydrogel in an aqueous solution to about 7% hydrogel in an aqueous solution, about 2% hydrogel in an aqueous solution to about 8% hydrogel in an aqueous solution, about 2% hydrogel in an aqueous solution to about 9% hydrogel in an aqueous solution, about 2% hydrogel in an aqueous solution to about 10% hydrogel in an aqueous solution, about 2% hydrogel in an aqueous solution to about 50% hydrogel in an aqueous solution, about 2% hydrogel in an aqueous solution to about 90% hydrogel in an aqueous solution, about 5% hydrogel in an aqueous solution to about 6% hydrogel in an aqueous solution, about 5% hydrogel in an aqueous solution to about 7% hydrogel in an aqueous solution, about 5% hydrogel in an aqueous solution to about 8% hydrogel in an aqueous solution, about 5% hydrogel in an aqueous solution to about 9% hydrogel in an aqueous solution, about 5% hydrogel in an aqueous solution to about 10% hydrogel in an aqueous solution, about 5% hydrogel in an aqueous solution to about 50% hydrogel in an aqueous solution, about 5% hydrogel in an aqueous solution to about 90% hydrogel in an aqueous solution, about 6% hydrogel in an aqueous solution to about 7% hydrogel in an aqueous solution, about 6% hydrogel in an aqueous solution to about 8% hydrogel in an aqueous solution, about 6% hydrogel in an aqueous solution to about 9% hydrogel in an aqueous solution, about 6% hydrogel in an aqueous solution to about 10% hydrogel in an aqueous solution, about 6% hydrogel in an aqueous solution to about 50% hydrogel in an aqueous solution, about 6% hydrogel in an aqueous solution to about 90% hydrogel in an aqueous solution, about 7% hydrogel in an aqueous solution to about 8% hydrogel in an aqueous solution, about 7% hydrogel in an aqueous solution to about 9% hydrogel in an aqueous solution, about 7% hydrogel in an aqueous solution to about 10% hydrogel in an aqueous solution, about 7% hydrogel in an aqueous solution to about 50% hydrogel in an aqueous solution, about 7% hydrogel in an aqueous solution to about 90% hydrogel in an aqueous solution, about 8% hydrogel in an aqueous solution to about 9% hydrogel in an aqueous solution, about 8% hydrogel in an aqueous solution to about 10% hydrogel in an aqueous solution, about 8% hydrogel in an aqueous solution to about 50% hydrogel in an aqueous solution, about 8% hydrogel in an aqueous solution to about 90% hydrogel in an aqueous solution, about 9% hydrogel in an aqueous solution to about 10% hydrogel in an aqueous solution, about 9% hydrogel in an aqueous solution to about 50% hydrogel in an aqueous solution, about 9% hydrogel in an aqueous solution to about 90% hydrogel in an aqueous solution, about 10% hydrogel in an aqueous solution to about 50% hydrogel in an aqueous solution, about 10% hydrogel in an aqueous solution to about 90% hydrogel in an aqueous solution, or about 50% hydrogel in an aqueous solution to about 90% hydrogel in an aqueous solution. In some embodiments, the hydrogel fibers are formed from a concentration of about 0.1% hydrogel in an aqueous solution, about 0.5% hydrogel in an aqueous solution, about 1% hydrogel in an aqueous solution, about 2% hydrogel in an aqueous solution, about 5% hydrogel in an aqueous solution, about 6% hydrogel in an aqueous solution, about 7% hydrogel in an aqueous solution, about 8% hydrogel in an aqueous solution, about 9% hydrogel in an aqueous solution, about 10% hydrogel in an aqueous solution, about 50% hydrogel in an aqueous solution, or about 90% hydrogel in an aqueous solution. In some embodiments, the hydrogel fibers are formed from a concentration of at least about 0.1% hydrogel in an aqueous solution, about 0.5% hydrogel in an aqueous solution, about 1% hydrogel in an aqueous solution, about 2% hydrogel in an aqueous solution, about 5% hydrogel in an aqueous solution, about 6% hydrogel in an aqueous solution, about 7% hydrogel in an aqueous solution, about 8% hydrogel in an aqueous solution, about 9% hydrogel in an aqueous solution, about 10% hydrogel in an aqueous solution, or about 50% hydrogel in an aqueous solution. In some embodiments, the hydrogel fibers are formed from a concentration of at most about 0.5% hydrogel in an aqueous solution, about 1% hydrogel in an aqueous solution, about 2% hydrogel in an aqueous solution, about 5% hydrogel in an aqueous solution, about 6% hydrogel in an aqueous solution, about 7% hydrogel in an aqueous solution, about 8% hydrogel in an aqueous solution, about 9% hydrogel in an aqueous solution, about 10% hydrogel in an aqueous solution, about 50% hydrogel in an aqueous solution, or about 90% hydrogel in an aqueous solution. In some embodiments, the hydrogel fibers are formed from a concentration of about 1% to about 12% (w/v) hydrogel in an aqueous solution. In some embodiments, the hydrogel fibers are formed from a concentration of about 2% to about 12% (w/v) hydrogel in an aqueous solution. In some embodiments, the hydrogel fibers are formed from a concentration of about 3% to about 12% (w/v) hydrogel in an aqueous solution. In some embodiments, the hydrogel fibers are formed from a concentration of about 4% to about 12% (w/v) hydrogel in an aqueous solution. In some embodiments, the hydrogel fibers are formed from a concentration of about 5% to about 12% (w/v) hydrogel in an aqueous solution.

In some embodiments, the hydrogel fibers comprise a hydrogel derived from gelatin. In some embodiments, the hydrogel fibers are formed from a concentration of about 1% to about 90% gelatin in an aqueous solution. In some embodiments, the hydrogel fibers are formed from a concentration of about 0.1% gelatin in an aqueous solution to about 90% gelatin in an aqueous solution. In some embodiments, the hydrogel fibers are formed from a concentration of about 0.1% gelatin in an aqueous solution to about 0.5% gelatin in an aqueous solution, about 0.1% gelatin in an aqueous solution to about 1% gelatin in an aqueous solution, about 0.1% gelatin in an aqueous solution to about 2% gelatin in an aqueous solution, about 0.1% gelatin in an aqueous solution to about 5% gelatin in an aqueous solution, about 0.1% gelatin in an aqueous solution to about 6% gelatin in an aqueous solution, about 0.1% gelatin in an aqueous solution to about 7% gelatin in an aqueous solution, about 0.1% gelatin in an aqueous solution to about 8% gelatin in an aqueous solution, about 0.1% gelatin in an aqueous solution to about 9% gelatin in an aqueous solution, about 0.1% gelatin in an aqueous solution to about 10% gelatin in an aqueous solution, about 0.1% gelatin in an aqueous solution to about 50% gelatin in an aqueous solution, about 0.1% gelatin in an aqueous solution to about 90% gelatin in an aqueous solution, about 0.5% gelatin in an aqueous solution to about 1% gelatin in an aqueous solution, about 0.5% gelatin in an aqueous solution to about 2% gelatin in an aqueous solution, about 0.5% gelatin in an aqueous solution to about 5% gelatin in an aqueous solution, about 0.5% gelatin in an aqueous solution to about 6% gelatin in an aqueous solution, about 0.5% gelatin in an aqueous solution to about 7% gelatin in an aqueous solution, about 0.5% gelatin in an aqueous solution to about 8% gelatin in an aqueous solution, about 0.5% gelatin in an aqueous solution to about 9% gelatin in an aqueous solution, about 0.5% gelatin in an aqueous solution to about 10% gelatin in an aqueous solution, about 0.5% gelatin in an aqueous solution to about 50% gelatin in an aqueous solution, about 0.5% gelatin in an aqueous solution to about 90% gelatin in an aqueous solution, about 1% gelatin in an aqueous solution to about 2% gelatin in an aqueous solution, about 1% gelatin in an aqueous solution to about 5% gelatin in an aqueous solution, about 1% gelatin in an aqueous solution to about 6% gelatin in an aqueous solution, about 1% gelatin in an aqueous solution to about 7% gelatin in an aqueous solution, about 1% gelatin in an aqueous solution to about 8% gelatin in an aqueous solution, about 1% gelatin in an aqueous solution to about 9% gelatin in an aqueous solution, about 1% gelatin in an aqueous solution to about 10% gelatin in an aqueous solution, about 1% gelatin in an aqueous solution to about 50% gelatin in an aqueous solution, about 1% gelatin in an aqueous solution to about 90% gelatin in an aqueous solution, about 2% gelatin in an aqueous solution to about 5% gelatin in an aqueous solution, about 2% gelatin in an aqueous solution to about 6% gelatin in an aqueous solution, about 2% gelatin in an aqueous solution to about 7% gelatin in an aqueous solution, about 2% gelatin in an aqueous solution to about 8% gelatin in an aqueous solution, about 2% gelatin in an aqueous solution to about 9% gelatin in an aqueous solution, about 2% gelatin in an aqueous solution to about 10% gelatin in an aqueous solution, about 2% gelatin in an aqueous solution to about 50% gelatin in an aqueous solution, about 2% gelatin in an aqueous solution to about 90% gelatin in an aqueous solution, about 5% gelatin in an aqueous solution to about 6% gelatin in an aqueous solution, about 5% gelatin in an aqueous solution to about 7% gelatin in an aqueous solution, about 5% gelatin in an aqueous solution to about 8% gelatin in an aqueous solution, about 5% gelatin in an aqueous solution to about 9% gelatin in an aqueous solution, about 5% gelatin in an aqueous solution to about 10% gelatin in an aqueous solution, about 5% gelatin in an aqueous solution to about 50% gelatin in an aqueous solution, about 5% gelatin in an aqueous solution to about 90% gelatin in an aqueous solution, about 6% gelatin in an aqueous solution to about 7% gelatin in an aqueous solution, about 6% gelatin in an aqueous solution to about 8% gelatin in an aqueous solution, about 6% gelatin in an aqueous solution to about 9% gelatin in an aqueous solution, about 6% gelatin in an aqueous solution to about 10% gelatin in an aqueous solution, about 6% gelatin in an aqueous solution to about 50% gelatin in an aqueous solution, about 6% gelatin in an aqueous solution to about 90% gelatin in an aqueous solution, about 7% gelatin in an aqueous solution to about 8% gelatin in an aqueous solution, about 7% gelatin in an aqueous solution to about 9% gelatin in an aqueous solution, about 7% gelatin in an aqueous solution to about 10% gelatin in an aqueous solution, about 7% gelatin in an aqueous solution to about 50% gelatin in an aqueous solution, about 7% gelatin in an aqueous solution to about 90% gelatin in an aqueous solution, about 8% gelatin in an aqueous solution to about 9% gelatin in an aqueous solution, about 8% gelatin in an aqueous solution to about 10% gelatin in an aqueous solution, about 8% gelatin in an aqueous solution to about 50% gelatin in an aqueous solution, about 8% gelatin in an aqueous solution to about 90% gelatin in an aqueous solution, about 9% gelatin in an aqueous solution to about 10% gelatin in an aqueous solution, about 9% gelatin in an aqueous solution to about 50% gelatin in an aqueous solution, about 9% gelatin in an aqueous solution to about 90% gelatin in an aqueous solution, about 10% gelatin in an aqueous solution to about 50% gelatin in an aqueous solution, about 10% gelatin in an aqueous solution to about 90% gelatin in an aqueous solution, or about 50% gelatin in an aqueous solution to about 90% gelatin in an aqueous solution. In some embodiments, the hydrogel fibers are formed from a concentration of about 0.1% gelatin in an aqueous solution, about 0.5% gelatin in an aqueous solution, about 1% gelatin in an aqueous solution, about 2% gelatin in an aqueous solution, about 5% gelatin in an aqueous solution, about 6% gelatin in an aqueous solution, about 7% gelatin in an aqueous solution, about 8% gelatin in an aqueous solution, about 9% gelatin in an aqueous solution, about 10% gelatin in an aqueous solution, about 50% gelatin in an aqueous solution, or about 90% gelatin in an aqueous solution. In some embodiments, the hydrogel fibers are formed from a concentration of at least about 0.1% gelatin in an aqueous solution, about 0.5% gelatin in an aqueous solution, about 1% gelatin in an aqueous solution, about 2% gelatin in an aqueous solution, about 5% gelatin in an aqueous solution, about 6% gelatin in an aqueous solution, about 7% gelatin in an aqueous solution, about 8% gelatin in an aqueous solution, about 9% gelatin in an aqueous solution, about 10% gelatin in an aqueous solution, or about 50% gelatin in an aqueous solution. In some embodiments, the hydrogel fibers are formed from a concentration of at most about 0.5% gelatin in an aqueous solution, about 1% gelatin in an aqueous solution, about 2% gelatin in an aqueous solution, about 5% gelatin in an aqueous solution, about 6% gelatin in an aqueous solution, about 7% gelatin in an aqueous solution, about 8% gelatin in an aqueous solution, about 9% gelatin in an aqueous solution, about 10% gelatin in an aqueous solution, about 50% gelatin in an aqueous solution, or about 90% gelatin in an aqueous solution.

In some embodiments, the method comprises forming the hydrogel fibers by dissolving a hydrogel or a combination of hydrogel described herein in an aqueous solution. In some embodiments, the method comprises dissolving the hydrogel in an aqueous solution at a temperature between about 4° C. to about 50° C. In some embodiments, the method comprises dissolving the hydrogel in an aqueous solution at a temperature between about 1° C. to about 75° C. In some embodiments, the method comprises dissolving the hydrogel in an aqueous solution at a temperature between about 1° C. to about 2° C., about 1° C. to about 4° C., about 1° C. to about 10° C., about 1° C. to about 15° C., about 1° C. to about 20° C., about 1° C. to about 30° C., about 1° C. to about 37° C., about 1° C. to about 40° C., about 1° C. to about 45° C., about 1° C. to about 50° C., about 1° C. to about 75° C., about 2° C. to about 4° C., about 2° C. to about 10° C., about 2° C. to about 15° C., about 2° C. to about 20° C., about 2° C. to about 30° C., about 2° C. to about 37° C., about 2° C. to about 40° C., about 2° C. to about 45° C., about 2° C. to about 50° C., about 2° C. to about 75° C., about 4° C. to about 10° C., about 4° C. to about 15° C., about 4° C. to about 20° C., about 4° C. to about 30° C., about 4° C. to about 37° C., about 4° C. to about 40° C., about 4° C. to about 45° C., about 4° C. to about 50° C., about 4° C. to about 75° C., about 10° C. to about 15° C., about 10° C. to about 20° C., about 10° C. to about 30° C., about 10° C. to about 37° C., about 10° C. to about 40° C., about 10° C. to about 45° C., about 10° C. to about 50° C., about 10° C. to about 75° C., about 15° C. to about 20° C., about 15° C. to about 30° C., about 15° C. to about 37° C., about 15° C. to about 40° C., about 15° C. to about 45° C., about 15° C. to about 50° C., about 15° C. to about 75° C., about 20° C. to about 30° C., about 20° C. to about 37° C., about 20° C. to about 40° C., about 20° C. to about 45° C., about 20° C. to about 50° C., about 20° C. to about 75° C., about 30° C. to about 37° C., about 30° C. to about 40° C., about 30° C. to about 45° C., about 30° C. to about 50° C., about 30° C. to about 75° C., about 37° C. to about 40° C., about 37° C. to about 45° C., about 37° C. to about 50° C., about 37° C. to about 75° C., about 40° C. to about 45° C., about 40° C. to about 50° C., about 40° C. to about 75° C., about 45° C. to about 50° C., about 45° C. to about 75° C., or about 50° C. to about 75° C. In some embodiments, the method comprises dissolving the hydrogel in an aqueous solution at a temperature between about 1° C., about 2° C., about 4° C., about 10° C., about 15° C., about 20° C., about 30° C., about 37° C., about 40° C., about 45° C., about 50° C., or about 75° C. In some embodiments, the method comprises dissolving the hydrogel in an aqueous solution at a temperature between at least about 1° C., about 2° C., about 4° C., about 10° C., about 15° C., about 20° C., about 30° C., about 37° C., about 40° C., about 45° C., or about 50° C. In some embodiments, the method comprises dissolving the hydrogel in an aqueous solution at a temperature between at most about 2° C., about 4° C., about 10° C., about 15° C., about 20° C., about 30° C., about 37° C., about 40° C., about 45° C., about 50° C., or about 75° C.

In some embodiments, the method comprises forming the hydrogel fibers by filling a assembly fitted with a mesh with micro-sized apertures with liquid hydrogel, allowing the liquid hydrogel to cool and solidify to obtain solid hydrogel, and extruding the solid hydrogel through the fitted assembly mesh to obtain hydrogel fibers. In some embodiments, the assembly comprises a vacuum assembly. In some embodiments, the assembly is a vacuum assembly. In some embodiments, the assembly is an extruder. In some embodiments, the assembly chamber pressure is from about 5 millitorr to about 4000 millitorr. In some embodiments, the assembly chamber pressure is from about 1 millitorr to about 5,000 millitorr. In some embodiments, the assembly chamber pressure is from about 1 millitorr to about 5 millitorr, about 1 millitorr to about 10 millitorr, about 1 millitorr to about 100 millitorr, about 1 millitorr to about 200 millitorr, about 1 millitorr to about 400 millitorr, about 1 millitorr to about 500 millitorr, about 1 millitorr to about 1,000 millitorr, about 1 millitorr to about 2,000 millitorr, about 1 millitorr to about 3,000 millitorr, about 1 millitorr to about 4,000 millitorr, about 1 millitorr to about 5,000 millitorr, about 5 millitorr to about 10 millitorr, about 5 millitorr to about 100 millitorr, about 5 millitorr to about 200 millitorr, about 5 millitorr to about 400 millitorr, about 5 millitorr to about 500 millitorr, about 5 millitorr to about 1,000 millitorr, about 5 millitorr to about 2,000 millitorr, about 5 millitorr to about 3,000 millitorr, about 5 millitorr to about 4,000 millitorr, about 5 millitorr to about 5,000 millitorr, about 10 millitorr to about 100 millitorr, about 10 millitorr to about 200 millitorr, about 10 millitorr to about 400 millitorr, about 10 millitorr to about 500 millitorr, about 10 millitorr to about 1,000 millitorr, about 10 millitorr to about 2,000 millitorr, about 10 millitorr to about 3,000 millitorr, about 10 millitorr to about 4,000 millitorr, about 10 millitorr to about 5,000 millitorr, about 100 millitorr to about 200 millitorr, about 100 millitorr to about 400 millitorr, about 100 millitorr to about 500 millitorr, about 100 millitorr to about 1,000 millitorr, about 100 millitorr to about 2,000 millitorr, about 100 millitorr to about 3,000 millitorr, about 100 millitorr to about 4,000 millitorr, about 100 millitorr to about 5,000 millitorr, about 200 millitorr to about 400 millitorr, about 200 millitorr to about 500 millitorr, about 200 millitorr to about 1,000 millitorr, about 200 millitorr to about 2,000 millitorr, about 200 millitorr to about 3,000 millitorr, about 200 millitorr to about 4,000 millitorr, about 200 millitorr to about 5,000 millitorr, about 400 millitorr to about 500 millitorr, about 400 millitorr to about 1,000 millitorr, about 400 millitorr to about 2,000 millitorr, about 400 millitorr to about 3,000 millitorr, about 400 millitorr to about 4,000 millitorr, about 400 millitorr to about 5,000 millitorr, about 500 millitorr to about 1,000 millitorr, about 500 millitorr to about 2,000 millitorr, about 500 millitorr to about 3,000 millitorr, about 500 millitorr to about 4,000 millitorr, about 500 millitorr to about 5,000 millitorr, about 1,000 millitorr to about 2,000 millitorr, about 1,000 millitorr to about 3,000 millitorr, about 1,000 millitorr to about 4,000 millitorr, about 1,000 millitorr to about 5,000 millitorr, about 2,000 millitorr to about 3,000 millitorr, about 2,000 millitorr to about 4,000 millitorr, about 2,000 millitorr to about 5,000 millitorr, about 3,000 millitorr to about 4,000 millitorr, about 3,000 millitorr to about 5,000 millitorr, or about 4,000 millitorr to about 5,000 millitorr. In some embodiments, the assembly chamber pressure is from about 1 millitorr, about 5 millitorr, about 10 millitorr, about 100 millitorr, about 200 millitorr, about 400 millitorr, about 500 millitorr, about 1,000 millitorr, about 2,000 millitorr, about 3,000 millitorr, about 4,000 millitorr, or about 5,000 millitorr. In some embodiments, the assembly chamber pressure is from at least about 1 millitorr, about 5 millitorr, about 10 millitorr, about 100 millitorr, about 200 millitorr, about 400 millitorr, about 500 millitorr, about 1,000 millitorr, about 2,000 millitorr, about 3,000 millitorr, or about 4,000 millitorr. In some embodiments, the assembly chamber pressure is from at most about 5 millitorr, about 10 millitorr, about 100 millitorr, about 200 millitorr, about 400 millitorr, about 500 millitorr, about 1,000 millitorr, about 2,000 millitorr, about 3,000 millitorr, about 4,000 millitorr, or about 5,000 millitorr.

In some embodiments, the method comprises forming the hydrogel fibers by extrusion. In some embodiments, the method comprises crosslinking the hydrogel fibers comprising: suspending the hydrogel fibers in an aqueous solution comprising at least one crosslinking reagent to obtain suspended hydrogel fibers; and maintaining the suspended hydrogel fibers for at least 6 hours at a temperature from about 0° C. to about 37° C. to obtain crosslinked hydrogel fibers. In some embodiments, the crosslinked hydrogel fibers are obtained by maintaining the suspended hydrogel fibers from about 0° C. to about 50° C. In some embodiments, the crosslinked hydrogel fibers are obtained by maintaining the suspended hydrogel fibers from about 0° C. to about 5° C., about 0° C. to about 10° C., about 0° C. to about 15° C., about 0° C. to about 20° C., about 0° C. to about 25° C., about 0° C. to about 30° C., about 0° C. to about 35° C., about 0° C. to about 37° C., about 0° C. to about 40° C., about 0° C. to about 45° C., about 0° C. to about 50° C., about 5° C. to about 10° C., about 5° C. to about 15° C., about 5° C. to about 20° C., about 5° C. to about 25° C., about 5° C. to about 30° C., about 5° C. to about 35° C., about 5° C. to about 37° C., about 5° C. to about 40° C., about 5° C. to about 45° C., about 5° C. to about 50° C., about 10° C. to about 15° C., about 10° C. to about 20° C., about 10° C. to about 25° C., about 10° C. to about 30° C., about 10° C. to about 35° C., about 10° C. to about 37° C., about 10° C. to about 40° C., about 10° C. to about 45° C., about 10° C. to about 50° C., about 15° C. to about 20° C., about 15° C. to about 25° C., about 15° C. to about 30° C., about 15° C. to about 35° C., about 15° C. to about 37° C., about 15° C. to about 40° C., about 15° C. to about 45° C., about 15° C. to about 50° C., about 20° C. to about 25° C., about 20° C. to about 30° C., about 20° C. to about 35° C., about 20° C. to about 37° C., about 20° C. to about 40° C., about 20° C. to about 45° C., about 20° C. to about 50° C., about 25° C. to about 30° C., about 25° C. to about 35° C., about 25° C. to about 37° C., about 25° C. to about 40° C., about 25° C. to about 45° C., about 25° C. to about 50° C., about 30° C. to about 35° C., about 30° C. to about 37° C., about 30° C. to about 40° C., about 30° C. to about 45° C., about 30° C. to about 50° C., about 35° C. to about 37° C., about 35° C. to about 40° C., about 35° C. to about 45° C., about 35° C. to about 50° C., about 37° C. to about 40° C., about 37° C. to about 45° C., about 37° C. to about 50° C., about 40° C. to about 45° C., about 40° C. to about 50° C., or about 45° C. to about 50° C. In some embodiments, the crosslinked hydrogel fibers are obtained by maintaining the suspended hydrogel fibers from about 0° C., about 5° C., about 10° C., about 15° C., about 20° C., about 25° C., about 30° C., about 35° C., about 37° C., about 40° C., about 45° C., or about 50° C. In some embodiments, the crosslinked hydrogel fibers are obtained by maintaining the suspended hydrogel fibers from at least about 0° C., about 5° C., about 10° C., about 15° C., about 20° C., about 25° C., about 30° C., about 35° C., about 37° C., about 40° C., or about 45° C. In some embodiments, the crosslinked hydrogel fibers are obtained by maintaining the suspended hydrogel fibers from at most about 5° C., about 10° C., about 15° C., about 20° C., about 25° C., about 30° C., about 35° C., about 37° C., about 40° C., about 45° C., or about 50° C.

In some embodiments, In some embodiments, the crosslinking agent comprises homobifunctional crosslinking reagents, heterobifunctional crosslinking reagents, phenolic crosslinking reagents, alginic acid, di- or polycarboxylic crosslinkers, oxidized polysaccharides, condensation involving cystamine/cysteine, photoreactive crosslinking reagents, disuccinimidyl suberate (DSS), disuccinimidyl tartrate (DST), dithiobis succinimidyl propionate (DSP), sulfhydryl-to-sulfhydryl crosslinkers, bismaleimidoethane (BMOE), dithiobismaleimidoethane (DTME), m-Maleimidobenzoyl-N-hydroxysuccinimide ester (MDS), N-γ-Maleimidobutyryloxysuccinimide ester (GMBS), N-(ε-Maleimidocaproyloxy) succinimide ester (EMCS), N-(ε-Maleimidocaproyloxy) sulfo succinimide ester (sulfo-EMCS), aryl azides, N-((2-pyridyldithio)ethyl)-4-azidosalicylamide, diazirines, transglutaminase, genipin, N-hydroxysuccinimide (NHS) ester, 1-Ethyl-3-[3-dimethylaminopropyl]carbodiimide hydrochloride (EDC or EDAC), hexamethylenetetramine, glutaraldehyde, epoxy compounds, isocyanates, or any combination thereof.

In some embodiments, the suspended hydrogel fibers is maintained with the at least one crosslinking agent for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 36 hours, at least 48 hours, or at least 60 hours. In some embodiments, the suspended hydrogel fibers is maintained with the at least one crosslinking agent for no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, no more than 24 hours, no more than 36 hours, no more than 48 hours, or no more than 60 hours. In some embodiments, the suspended hydrogel fibers are maintained with at least one crosslinking agent at a temperature from about 0° C. to about 10° C. to obtain crosslinked hydrogel fibers. In some embodiments, the suspended hydrogel fibers are maintained with at least one crosslinking agent at a temperature from about 0° C. to about 37° C. In some embodiments, the suspended hydrogel fibers is maintained with the at least one crosslinking agent at a temperature from about 0° C. to about 1° C., about 0° C. to about 2° C., about 0° C. to about 3° C., about 0° C. to about 4° C., about 0° C. to about 5° C., about 0° C. to about 6° C., about 0° C. to about 7° C., about 0° C. to about 8° C., about 0° C. to about 9° C., about 0° C. to about 10° C., about 0° C. to about 15° C., about 0° C. to about 37° C., about 1° C. to about 2° C., about 1° C. to about 3° C., about 1° C. to about 4° C., about 1° C. to about 5° C., about 1° C. to about 6° C., about 1° C. to about 7° C., about 1° C. to about 8° C., about 1° C. to about 9° C., about 1° C. to about 10° C., about 1° C. to about 15° C., about 2° C. to about 3° C., about 2° C. to about 4° C., about 2° C. to about 5° C., about 2° C. to about 6° C., about 2° C. to about 7° C., about 2° C. to about 8° C., about 2° C. to about 9° C., about 2° C. to about 10° C., about 2° C. to about 15° C., about 2° C. to about 37° C., about 3° C. to about 4° C., about 3° C. to about 5° C., about 3° C. to about 6° C., about 3° C. to about 7° C., about 3° C. to about 8° C., about 3° C. to about 9° C., about 3° C. to about 10° C., about 3° C. to about 15° C., about 3° C. to about 37° C., about 4° C. to about 5° C., about 4° C. to about 6° C., about 4° C. to about 7° C., about 4° C. to about 8° C., about 4° C. to about 9° C., about 4° C. to about 10° C., about 4° C. to about 15° C., about 0° C. to about 37° C., about 5° C. to about 6° C., about 5° C. to about 7° C., about 5° C. to about 8° C., about 5° C. to about 9° C., about 5° C. to about 10° C., about 5° C. to about 15° C., about 5° C. to about 37° C., about 6° C. to about 7° C., about 6° C. to about 8° C., about 6° C. to about 9° C., about 6° C. to about 10° C., about 6° C. to about 15° C., about 6° C. to about 37° C., about 7° C. to about 8° C., about 7° C. to about 9° C., about 7° C. to about 10° C., about 7° C. to about 15° C., about 7° C. to about 37° C., about 8° C. to about 9° C., about 8° C. to about 10° C., about 8° C. to about 15° C., about 9° C. to about 10° C., about 9° C. to about 15° C., or about 10° C. to about 15° C., about 8° C. to about 37° C., about 9° C. to about 37° C., about 10° C. to about 37° C. In some embodiments, the suspended hydrogel fibers is maintained with the at least one crosslinking agent at a temperature from about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 20° C., about 25° C., about 30° C., or about 37° C. In some embodiments, the suspended hydrogel fibers is maintained with the at least one crosslinking agent at a temperature from at least about 0° C., about 1° C., about 2° C., about 3° C., about 4° C., about 5° C., about 6° C., about 7° C., about 8° C., about 9° C., or about 10° C. In some embodiments, the suspended hydrogel fibers is maintained with the at least one crosslinking agent at a temperature from at most about 1° C., about 2° C., about 3° C., about 4° C., about 5° C., about 6° C., about 7° C., about 8° C., about 9° C., about 10° C., about 15° C., about 20° C., about 25° C., about 30° C., or about 37° C.

In some embodiments, the method comprises crosslinking the hydrogel fibers to obtain crosslinked hydrogel fibers by shaker cultivating the suspended hydrogel fibers from about 50 revolutions per minute (RPM) to about 500 revolutions per minute (RPM). In some embodiments, the method comprises obtaining the crosslinked hydrogel fibers by shaker cultivating the suspended hydrogel fibers from about 10 RPM to about 1,500 RPM. In some embodiments, the method comprises obtaining the crosslinked hydrogel fibers by shaker cultivating the suspended hydrogel fibers from about 10 RPM to about 20 RPM, about 10 RPM to about 50 RPM, about 10 RPM to about 100 RPM, about 10 RPM to about 200 RPM, about 10 RPM to about 300 RPM, about 10 RPM to about 400 RPM, about 10 RPM to about 500 RPM, about 10 RPM to about 600 RPM, about 10 RPM to about 800 RPM, about 10 RPM to about 1,000 RPM, about 10 RPM to about 1,500 RPM, about 20 RPM to about 50 RPM, about 20 RPM to about 100 RPM, about 20 RPM to about 200 RPM, about 20 RPM to about 300 RPM, about 20 RPM to about 400 RPM, about 20 RPM to about 500 RPM, about 20 RPM to about 600 RPM, about 20 RPM to about 800 RPM, about 20 RPM to about 1,000 RPM, about 20 RPM to about 1,500 RPM, about 50 RPM to about 100 RPM, about 50 RPM to about 200 RPM, about 50 RPM to about 300 RPM, about 50 RPM to about 400 RPM, about 50 RPM to about 500 RPM, about 50 RPM to about 600 RPM, about 50 RPM to about 800 RPM, about 50 RPM to about 1,000 RPM, about 50 RPM to about 1,500 RPM, about 100 RPM to about 200 RPM, about 100 RPM to about 300 RPM, about 100 RPM to about 400 RPM, about 100 RPM to about 500 RPM, about 100 RPM to about 600 RPM, about 100 RPM to about 800 RPM, about 100 RPM to about 1,000 RPM, about 100 RPM to about 1,500 RPM, about 200 RPM to about 300 RPM, about 200 RPM to about 400 RPM, about 200 RPM to about 500 RPM, about 200 RPM to about 600 RPM, about 200 RPM to about 800 RPM, about 200 RPM to about 1,000 RPM, about 200 RPM to about 1,500 RPM, about 300 RPM to about 400 RPM, about 300 RPM to about 500 RPM, about 300 RPM to about 600 RPM, about 300 RPM to about 800 RPM, about 300 RPM to about 1,000 RPM, about 300 RPM to about 1,500 RPM, about 400 RPM to about 500 RPM, about 400 RPM to about 600 RPM, about 400 RPM to about 800 RPM, about 400 RPM to about 1,000 RPM, about 400 RPM to about 1,500 RPM, about 500 RPM to about 600 RPM, about 500 RPM to about 800 RPM, about 500 RPM to about 1,000 RPM, about 500 RPM to about 1,500 RPM, about 600 RPM to about 800 RPM, about 600 RPM to about 1,000 RPM, about 600 RPM to about 1,500 RPM, about 800 RPM to about 1,000 RPM, about 800 RPM to about 1,500 RPM, or about 1,000 RPM to about 1,500 RPM. In some embodiments, the method comprises obtaining the crosslinked hydrogel fibers by shaker cultivating the suspended hydrogel fibers from about 10 RPM, about 20 RPM, about 50 RPM, about 100 RPM, about 200 RPM, about 300 RPM, about 400 RPM, about 500 RPM, about 600 RPM, about 800 RPM, about 1,000 RPM, or about 1,500 RPM. In some embodiments, the method comprises obtaining the crosslinked hydrogel fibers by shaker cultivating the suspended hydrogel fibers from at least about 10 RPM, about 20 RPM, about 50 RPM, about 100 RPM, about 200 RPM, about 300 RPM, about 400 RPM, about 500 RPM, about 600 RPM, about 800 RPM, or about 1,000 RPM. In some embodiments, the method comprises obtaining the crosslinked hydrogel fibers by shaker cultivating the suspended hydrogel fibers from at most about 20 RPM, about 50 RPM, about 100 RPM, about 200 RPM, about 300 RPM, about 400 RPM, about 500 RPM, about 600 RPM, about 800 RPM, about 1,000 RPM, or about 1,500 RPM.

In some embodiments, the method comprises crosslinking the hydrogel fibers by a photo-crosslinking reaction. In some embodiments, the ultraviolet light comprises a wavelength of about 10 nanometers to about 1,500 nanometers. In some embodiments, the ultraviolet light comprises a wavelength of about 10 nanometers to about 20 nanometers, about 10 nanometers to about 50 nanometers, about 10 nanometers to about 100 nanometers, about 10 nanometers to about 200 nanometers, about 10 nanometers to about 300 nanometers, about 10 nanometers to about 400 nanometers, about 10 nanometers to about 500 nanometers, about 10 nanometers to about 600 nanometers, about 10 nanometers to about 800 nanometers, about 10 nanometers to about 1,000 nanometers, about 10 nanometers to about 1,500 nanometers, about 20 nanometers to about 50 nanometers, about 20 nanometers to about 100 nanometers, about 20 nanometers to about 200 nanometers, about 20 nanometers to about 300 nanometers, about 20 nanometers to about 400 nanometers, about 20 nanometers to about 500 nanometers, about 20 nanometers to about 600 nanometers, about 20 nanometers to about 800 nanometers, about 20 nanometers to about 1,000 nanometers, about 20 nanometers to about 1,500 nanometers, about 50 nanometers to about 100 nanometers, about 50 nanometers to about 200 nanometers, about 50 nanometers to about 300 nanometers, about 50 nanometers to about 400 nanometers, about 50 nanometers to about 500 nanometers, about 50 nanometers to about 600 nanometers, about 50 nanometers to about 800 nanometers, about 50 nanometers to about 1,000 nanometers, about 50 nanometers to about 1,500 nanometers, about 100 nanometers to about 200 nanometers, about 100 nanometers to about 300 nanometers, about 100 nanometers to about 400 nanometers, about 100 nanometers to about 500 nanometers, about 100 nanometers to about 600 nanometers, about 100 nanometers to about 800 nanometers, about 100 nanometers to about 1,000 nanometers, about 100 nanometers to about 1,500 nanometers, about 200 nanometers to about 300 nanometers, about 200 nanometers to about 400 nanometers, about 200 nanometers to about 500 nanometers, about 200 nanometers to about 600 nanometers, about 200 nanometers to about 800 nanometers, about 200 nanometers to about 1,000 nanometers, about 200 nanometers to about 1,500 nanometers, about 300 nanometers to about 400 nanometers, about 300 nanometers to about 500 nanometers, about 300 nanometers to about 600 nanometers, about 300 nanometers to about 800 nanometers, about 300 nanometers to about 1,000 nanometers, about 300 nanometers to about 1,500 nanometers, about 400 nanometers to about 500 nanometers, about 400 nanometers to about 600 nanometers, about 400 nanometers to about 800 nanometers, about 400 nanometers to about 1,000 nanometers, about 400 nanometers to about 1,500 nanometers, about 500 nanometers to about 600 nanometers, about 500 nanometers to about 800 nanometers, about 500 nanometers to about 1,000 nanometers, about 500 nanometers to about 1,500 nanometers, about 600 nanometers to about 800 nanometers, about 600 nanometers to about 1,000 nanometers, about 600 nanometers to about 1,500 nanometers, about 800 nanometers to about 1,000 nanometers, about 800 nanometers to about 1,500 nanometers, or about 1,000 nanometers to about 1,500 nanometers. In some embodiments, the ultraviolet light comprises a wavelength of about 10 nanometers, about 20 nanometers, about 50 nanometers, about 100 nanometers, about 200 nanometers, about 300 nanometers, about 400 nanometers, about 500 nanometers, about 600 nanometers, about 800 nanometers, about 1,000 nanometers, or about 1,500 nanometers. In some embodiments, the ultraviolet light comprises a wavelength of at least about 10 nanometers, about 20 nanometers, about 50 nanometers, about 100 nanometers, about 200 nanometers, about 300 nanometers, about 400 nanometers, about 500 nanometers, about 600 nanometers, about 800 nanometers, or about 1,000 nanometers. In some embodiments, the ultraviolet light comprises a wavelength of at most about 20 nanometers, about 50 nanometers, about 100 nanometers, about 200 nanometers, about 300 nanometers, about 400 nanometers, about 500 nanometers, about 600 nanometers, about 800 nanometers, about 1,000 nanometers, or about 1,500 nanometers.

In some embodiments, the method comprises lyophilizing the crosslinked hydrogel fibers to obtain crosslinked lyophilized hydrogel fibers comprises: freezing the crosslinked hydrogel fibers at a first temperature sufficient to transform the water in crosslinked hydrogel fibers from liquid state to solid state; and drying the crosslinked hydrogel fibers at a second temperature sufficient to remove the water by sublimation from the crosslinked hydrogel fibers. In some embodiments, the first temperature is from about −100° C. to about −10° C. In some embodiments, the first temperature is from about −100° C. to about −90° C., about −100° C. to about −80° C., about −100° C. to about −70° C., about −100° C. to about −60° C., about −100° C. to about −50° C., about −100° C. to about −40° C., about −100° C. to about −30° C., about −100° C. to about −20° C., about −100° C. to about −10° C., about −90° C. to about −80° C., about −90° C. to about −70° C., about −90° C. to about −60° C., about −90° C. to about −50° C., about −90° C. to about −40° C., about −90° C. to about −30° C., about −90° C. to about −20° C., about −90° C. to about −10° C., about −80° C. to about −70° C., about −80° C. to about −60° C., about −80° C. to about −50° C., about −80° C. to about −40° C., about −80° C. to about −30° C., about −80° C. to about −20° C., about −80° C. to about −10° C., about −70° C. to about −60° C., about −70° C. to about −50° C., about −70° C. to about −40° C., about −70° C. to about −30° C., about −70° C. to about −20° C., about −70° C. to about −10° C., about −60° C. to about −50° C., about −60° C. to about −40° C., about −60° C. to about −30° C., about −60° C. to about −20° C., about −60° C. to about −10° C., about −50° C. to about −40° C., about −50° C. to about −30° C., about −50° C. to about −20° C., about −50° C. to about −10° C., about −40° C. to about −30° C., about −40° C. to about −20° C., about −40° C. to about −10° C., about −30° C. to about −20° C., about −30° C. to about −10° C., or about −20° C. to about −10° C. In some embodiments, the first temperature is from about −100° C., about −90° C., about −80° C., about −70° C., about −60° C., about −50° C., about −40° C., about −30° C., about −20° C., or about −10° C. In some embodiments, the first temperature is from at least about −100° C., about −90° C., about −80° C., about −70° C., about −60° C., about −50° C., about −40° C., about −30° C., or about −20° C. In some embodiments, the first temperature is from at most about −90° C., about −80° C., about −70° C., about −60° C., about −50° C., about −40° C., about −30° C., about −20° C., or about −10° C. In some embodiments, the second temperature is from about −60° C. to about 50° C. In some embodiments, the second temperature is from about −60° C. to about −50° C., about −60° C. to about −40° C., about −60° C. to about −30° C., about −60° C. to about −20° C., about −60° C. to about −10° C., about −60° C. to about 0° C., about −60° C. to about 10° C., about −60° C. to about 20° C., about −60° C. to about 30° C., about −60° C. to about 40° C., about −60° C. to about 50° C., about-50° C. to about −40° C., about −50° C. to about −30° C., about −50° C. to about −20° C., about −50° C. to about −10° C., about −50° C. to about 0° C., about −50° C. to about 10° C., about −50° C. to about 20° C., about −50° C. to about 30° C., about −50° C. to about 40° C., about −50° C. to about 50° C., about −40° C. to about −30° C., about −40° C. to about −20° C., about −40° C. to about −10° C., about −40° C. to about 0° C., about −40° C. to about 10° C., about −40° C. to about 20° C., about −40° C. to about 30° C., about −40° C. to about 40° C., about −40° C. to about 50° C., about −30° C. to about-20° C., about −30° C. to about −10° C., about −30° C. to about 0° C., about −30° C. to about 10° C., about −30° C. to about 20° C., about −30° C. to about 30° C., about −30° C. to about 40° C., about-30° C. to about 50° C., about −20° C. to about −10° C., about −20° C. to about 0° C., about −20° C. to about 10° C., about −20° C. to about 20° C., about −20° C. to about 30° C., about −20° C. to about 40° C., about −20° C. to about 50° C., about −10° C. to about 0° C., about −10° C. to about 10° C., about −10° C. to about 20° C., about −10° C. to about 30° C., about −10° C. to about 40° C., about −10° C. to about 50° C., about 0° C. to about 10° C., about 0° C. to about 20° C., about 0° C. to about 30° C., about 0° C. to about 40° C., about 0° C. to about 50° C., about 10° C. to about 20° C., about 10° C. to about 30° C., about 10° C. to about 40° C., about 10° C. to about 50° C., about 20° C. to about 30° C., about 20° C. to about 40° C., about 20° C. to about 50° C., about 30° C. to about 40° C., about 30° C. to about 50° C., or about 40° C. to about 50° C. In some embodiments, the second temperature is from about −60° C., about −50° C., about −40° C., about −30° C., about −20° C., about −10° C., about 0° C., about 10° C., about 20° C., about 30° C., about 40° C., or about 50° C. In some embodiments, the second temperature is from at least about −60° C., about −50° C., about −40° C., about −30° C., about −20° C., about −10° C., about 0° C., about 10° C., about 20° C., about 30° C., or about 40° C. In some embodiments, the second temperature is from at most about −50° C., about −40° C., about −30° C., about −20° C., about −10° C., about 0° C., about 10° C., about 20° C., about 30° C., about 40° C., or about 50° C. In some embodiments, the method comprises washing the crosslinked hydrogel fibers or the lyophilized crosslinked hydrogel fibers.

In some embodiments, the method comprises drying the crosslinked hydrogel fibers in a vacuum chamber. In some embodiments, the method comprises drying the crosslinked hydrogel fibers in a vacuum chamber at a pressure from about 1 millitorr to about 5,000 millitorr. In some embodiments, the method comprises drying the crosslinked hydrogel fibers in a vacuum chamber at a pressure from about 1 millitorr to about 2 millitorr, about 1 millitorr to about 5 millitorr, about 1 millitorr to about 10 millitorr, about 1 millitorr to about 100 millitorr, about 1 millitorr to about 300 millitorr, about 1 millitorr to about 500 millitorr, about 1 millitorr to about 1,000 millitorr, about 1 millitorr to about 2,000 millitorr, about 1 millitorr to about 3,000 millitorr, about 1 millitorr to about 4,000 millitorr, about 1 millitorr to about 5,000 millitorr, about 2 millitorr to about 5 millitorr, about 2 millitorr to about 10 millitorr, about 2 millitorr to about 100 millitorr, about 2 millitorr to about 300 millitorr, about 2 millitorr to about 500 millitorr, about 2 millitorr to about 1,000 millitorr, about 2 millitorr to about 2,000 millitorr, about 2 millitorr to about 3,000 millitorr, about 2 millitorr to about 4,000 millitorr, about 2 millitorr to about 5,000 millitorr, about 5 millitorr to about 10 millitorr, about 5 millitorr to about 100 millitorr, about 5 millitorr to about 300 millitorr, about 5 millitorr to about 500 millitorr, about 5 millitorr to about 1,000 millitorr, about 5 millitorr to about 2,000 millitorr, about 5 millitorr to about 3,000 millitorr, about 5 millitorr to about 4,000 millitorr, about 5 millitorr to about 5,000 millitorr, about 10 millitorr to about 100 millitorr, about 10 millitorr to about 300 millitorr, about 10 millitorr to about 500 millitorr, about 10 millitorr to about 1,000 millitorr, about 10 millitorr to about 2,000 millitorr, about 10 millitorr to about 3,000 millitorr, about 10 millitorr to about 4,000 millitorr, about 10 millitorr to about 5,000 millitorr, about 100 millitorr to about 300 millitorr, about 100 millitorr to about 500 millitorr, about 100 millitorr to about 1,000 millitorr, about 100 millitorr to about 2,000 millitorr, about 100 millitorr to about 3,000 millitorr, about 100 millitorr to about 4,000 millitorr, about 100 millitorr to about 5,000 millitorr, about 300 millitorr to about 500 millitorr, about 300 millitorr to about 1,000 millitorr, about 300 millitorr to about 2,000 millitorr, about 300 millitorr to about 3,000 millitorr, about 300 millitorr to about 4,000 millitorr, about 300 millitorr to about 5,000 millitorr, about 500 millitorr to about 1,000 millitorr, about 500 millitorr to about 2,000 millitorr, about 500 millitorr to about 3,000 millitorr, about 500 millitorr to about 4,000 millitorr, about 500 millitorr to about 5,000 millitorr, about 1,000 millitorr to about 2,000 millitorr, about 1,000 millitorr to about 3,000 millitorr, about 1,000 millitorr to about 4,000 millitorr, about 1,000 millitorr to about 5,000 millitorr, about 2,000 millitorr to about 3,000 millitorr, about 2,000 millitorr to about 4,000 millitorr, about 2,000 millitorr to about 5,000 millitorr, about 3,000 millitorr to about 4,000 millitorr, about 3,000 millitorr to about 5,000 millitorr, or about 4,000 millitorr to about 5,000 millitorr. In some embodiments, the method comprises drying the crosslinked hydrogel fibers in a vacuum chamber at a pressure from about 1 millitorr, about 2 millitorr, about 5 millitorr, about 10 millitorr, about 100 millitorr, about 300 millitorr, about 500 millitorr, about 1,000 millitorr, about 2,000 millitorr, about 3,000 millitorr, about 4,000 millitorr, or about 5,000 millitorr. In some embodiments, the method comprises drying the crosslinked hydrogel fibers in a vacuum chamber at a pressure from at least about 1 millitorr, about 2 millitorr, about 5 millitorr, about 10 millitorr, about 100 millitorr, about 300 millitorr, about 500 millitorr, about 1,000 millitorr, about 2,000 millitorr, about 3,000 millitorr, or about 4,000 millitorr. In some embodiments, the method comprises drying the crosslinked hydrogel fibers in a vacuum chamber at a pressure from at most about 2 millitorr, about 5 millitorr, about 10 millitorr, about 100 millitorr, about 300 millitorr, about 500 millitorr, about 1,000 millitorr, about 2,000 millitorr, about 3,000 millitorr, about 4,000 millitorr, or about 5,000 millitorr.

In some embodiments, the method comprises sterilizing the crosslinked hydrogel fibers or the lyophilized crosslinked hydrogel fibers. In some embodiments, the method comprises sterilizing by heating or irradiating the lyophilized crosslinked hydrogel fibers; or immersing the lyophilized crosslinked hydrogel fibers in alcohol-based soaking solution. In some embodiments, the sterilization by heating comprises heating the crosslinked hydrogel fibers or the lyophilized crosslinked hydrogel fibers to a temperature about 30° C. to about 150° C. In some embodiments, the sterilization by heating comprises heating the crosslinked hydrogel fibers or the lyophilized crosslinked hydrogel fibers to a temperature about 30° C. to about 37° C., about 30° C. to about 40° C., about 30° C. to about 50° C., about 30° C. to about 60° C., about 30° C. to about 70° C., about 30° C. to about 80° C., about 30° C. to about 90° C., about 30° C. to about 100° C., about 30° C. to about 121° C., about 30° C. to about 130° C., about 30° C. to about 150° C., about 37° C. to about 40° C., about 37° C. to about 50° C., about 37° C. to about 60° C., about 37° C. to about 70° C., about 37° C. to about 80° C., about 37° C. to about 90° C., about 37° C. to about 100° C., about 37° C. to about 121° C., about 37° C. to about 130° C., about 37° C. to about 150° C., about 40° C. to about 50° C., about 40° C. to about 60° C., about 40° C. to about 70° C., about 40° C. to about 80° C., about 40° C. to about 90° C., about 40° C. to about 100° C., about 40° C. to about 121° C., about 40° C. to about 130° C., about 40° C. to about 150° C., about 50° C. to about 60° C., about 50° C. to about 70° C., about 50° C. to about 80° C., about 50° C. to about 90° C., about 50° C. to about 100° C., about 50° C. to about 121° C., about 50° C. to about 130° C., about 50° C. to about 150° C., about 60° C. to about 70° C., about 60° C. to about 80° C., about 60° C. to about 90° C., about 60° C. to about 100° C., about 60° C. to about 121° C., about 60° C. to about 130° C., about 60° C. to about 150° C., about 70° C. to about 80° C., about 70° C. to about 90° C., about 70° C. to about 100° C., about 70° C. to about 121° C., about 70° C. to about 130° C., about 70° C. to about 150° C., about 80° C. to about 90° C., about 80° C. to about 100° C., about 80° C. to about 121° C., about 80° C. to about 130° C., about 80° C. to about 150° C., about 90° C. to about 100° C., about 90° C. to about 121° C., about 90° C. to about 130° C., about 90° C. to about 150° C., about 100° C. to about 121° C., about 100° C. to about 130° C., about 100° C. to about 150° C., about 121° C. to about 130° C., about 121° C. to about 150° C., or about 130° C. to about 150° C. In some embodiments, the sterilization by heating comprises heating the crosslinked hydrogel fibers or the lyophilized crosslinked hydrogel fibers to a temperature about 30° C., about 37° C., about 40° C., about 50° C., about 60° C., about 70° C., about 80° C., about 90° C., about 100° C., about 121° C., about 130° C., or about 150° C. In some embodiments, the sterilization by heating comprises heating the crosslinked hydrogel fibers or the lyophilized crosslinked hydrogel fibers to a temperature at least about 30° C., about 37° C., about 40° C., about 50° C., about 60° C., about 70° C., about 80° C., about 90° C., about 100° C., about 121° C., or about 130° C. In some embodiments, the sterilization by heating comprises heating the crosslinked hydrogel fibers or the lyophilized crosslinked hydrogel fibers to a temperature at most about 37° C., about 40° C., about 50° C., about 60° C., about 70° C., about 80° C., about 90° C., about 100° C., about 121° C., about 130° C., or about 150° C. In some embodiments, the sterilization by irradiation comprises contacting the crosslinked hydrogel fibers or the lyophilized crosslinked hydrogel fibers with UV radiation.

In some embodiments, the hydrogel fibers obtained by the method described herein comprise a flexible dissolution rate from about 3 minutes to about 100 days. In some embodiments, the hydrogel fibers obtained by the method described herein comprise a flexible dissolution rate from about 3 minutes to about 60 minutes. In some embodiments, the hydrogel fibers obtained by the method described herein comprise a flexible dissolution rate from about 3 minutes to about 5 minutes, about 3 minutes to about 10 minutes, about 3 minutes to about 20 minutes, about 3 minutes to about 30 minutes, about 3 minutes to about 40 minutes, about 3 minutes to about 50 minutes, about 3 minutes to about 60 minutes, about 5 minutes to about 10 minutes, about 5 minutes to about 20 minutes, about 5 minutes to about 30 minutes, about 5 minutes to about 40 minutes, about 5 minutes to about 50 minutes, about 5 minutes to about 60 minutes, about 10 minutes to about 20 minutes, about 10 minutes to about 30 minutes, about 10 minutes to about 40 minutes, about 10 minutes to about 50 minutes, about 10 minutes to about 60 minutes, about 20 minutes to about 30 minutes, about 20 minutes to about 40 minutes, about 20 minutes to about 50 minutes, about 20 minutes to about 60 minutes, about 30 minutes to about 40 minutes, about 30 minutes to about 50 minutes, about 30 minutes to about 60 minutes, about 40 minutes to about 50 minutes, about 40 minutes to about 60 minutes, or about 50 minutes to about 60 minutes. In some embodiments, the hydrogel fibers obtained by the method described herein comprise a flexible dissolution rate from about 3 minutes, about 5 minutes, about 10 minutes, about 20 minutes, about 30 minutes, about 40 minutes, about 50 minutes, or about 60 minutes. In some embodiments, the hydrogel fibers obtained by the method described herein comprise a flexible dissolution rate from at least about 3 minutes, about 5 minutes, about 10 minutes, about 20 minutes, about 30 minutes, about 40 minutes, or about 50 minutes. In some embodiments, the hydrogel fibers obtained by the method described herein comprise a flexible dissolution rate from at most about 5 minutes, about 10 minutes, about 20 minutes, about 30 minutes, about 40 minutes, about 50 minutes, or about 60 minutes. In some embodiments, the hydrogel fibers obtained by the method described herein comprise a flexible dissolution rate from about 1 hour to about 72 hours. In some embodiments, the hydrogel fibers obtained by the method described herein comprise a flexible dissolution rate from about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 12 hours, about 1 hour to about 24 hours, about 1 hour to about 48 hours, about 1 hour to about 72 hours, about 2 hours to about 3 hours, about 2 hours to about 4 hours, about 2 hours to about 5 hours, about 2 hours to about 6 hours, about 2 hours to about 7 hours, about 2 hours to about 8 hours, about 2 hours to about 12 hours, about 2 hours to about 24 hours, about 2 hours to about 48 hours, about 2 hours to about 72 hours, about 3 hours to about 4 hours, about 3 hours to about 5 hours, about 3 hours to about 6 hours, about 3 hours to about 7 hours, about 3 hours to about 8 hours, about 3 hours to about 12 hours, about 3 hours to about 24 hours, about 3 hours to about 48 hours, about 3 hours to about 72 hours, about 4 hours to about 5 hours, about 4 hours to about 6 hours, about 4 hours to about 7 hours, about 4 hours to about 8 hours, about 4 hours to about 12 hours, about 4 hours to about 24 hours, about 4 hours to about 48 hours, about 4 hours to about 72 hours, about 5 hours to about 6 hours, about 5 hours to about 7 hours, about 5 hours to about 8 hours, about 5 hours to about 12 hours, about 5 hours to about 24 hours, about 5 hours to about 48 hours, about 5 hours to about 72 hours, about 6 hours to about 7 hours, about 6 hours to about 8 hours, about 6 hours to about 12 hours, about 6 hours to about 24 hours, about 6 hours to about 48 hours, about 6 hours to about 72 hours, about 7 hours to about 8 hours, about 7 hours to about 12 hours, about 7 hours to about 24 hours, about 7 hours to about 48 hours, about 7 hours to about 72 hours, about 8 hours to about 12 hours, about 8 hours to about 24 hours, about 8 hours to about 48 hours, about 8 hours to about 72 hours, about 12 hours to about 24 hours, about 12 hours to about 48 hours, about 12 hours to about 72 hours, about 24 hours to about 48 hours, about 24 hours to about 72 hours, or about 48 hours to about 72 hours. In some embodiments, the hydrogel fibers obtained by the method described herein comprise a flexible dissolution rate from about 1 hour, about 2 hours, about 3 hours, about 4 hours, about 5 hours, about 6 hours, about 7 hours, about 8 hours, about 12 hours, about 24 hours, about 48 hours, or about 72 hours. In some embodiments, the hydrogel fibers obtained by the method described herein comprise a flexible dissolution rate from at least about 1 hour, about 2 hours, about 3 hours, about 4 hours, about 5 hours, about 6 hours, about 7 hours, about 8 hours, about 12 hours, about 24 hours, or about 48 hours. In some embodiments, the hydrogel fibers obtained by the method described herein comprise a flexible dissolution rate from at most about 2 hours, about 3 hours, about 4 hours, about 5 hours, about 6 hours, about 7 hours, about 8 hours, about 12 hours, about 24 hours, about 48 hours, or about 72 hours. In some embodiments, the hydrogel fibers obtained by the method described herein comprise a flexible dissolution rate from about 1 day to about 120 days. In some embodiments, the hydrogel fibers obtained by the method described herein comprise a flexible dissolution rate from about 1 day to about 2 days, about 1 day to about 3 days, about 1 day to about 4 days, about 1 day to about 5 days, about 1 day to about 8 days, about 1 day to about 10 days, about 1 day to about 20 days, about 1 day to about 30 days, about 1 day to about 50 days, about 1 day to about 100 days, about 1 day to about 120 days, about 2 days to about 3 days, about 2 days to about 4 days, about 2 days to about 5 days, about 2 days to about 8 days, about 2 days to about 10 days, about 2 days to about 20 days, about 2 days to about 30 days, about 2 days to about 50 days, about 2 days to about 100 days, about 2 days to about 120 days, about 3 days to about 4 days, about 3 days to about 5 days, about 3 days to about 8 days, about 3 days to about 10 days, about 3 days to about 20 days, about 3 days to about 30 days, about 3 days to about 50 days, about 3 days to about 100 days, about 3 days to about 120 days, about 4 days to about 5 days, about 4 days to about 8 days, about 4 days to about 10 days, about 4 days to about 20 days, about 4 days to about 30 days, about 4 days to about 50 days, about 4 days to about 100 days, about 4 days to about 120 days, about 5 days to about 8 days, about 5 days to about 10 days, about 5 days to about 20 days, about 5 days to about 30 days, about 5 days to about 50 days, about 5 days to about 100 days, about 5 days to about 120 days, about 8 days to about 10 days, about 8 days to about 20 days, about 8 days to about 30 days, about 8 days to about 50 days, about 8 days to about 100 days, about 8 days to about 120 days, about 10 days to about 20 days, about 10 days to about 30 days, about 10 days to about 50 days, about 10 days to about 100 days, about 10 days to about 120 days, about 20 days to about 30 days, about 20 days to about 50 days, about 20 days to about 100 days, about 20 days to about 120 days, about 30 days to about 50 days, about 30 days to about 100 days, about 30 days to about 120 days, about 50 days to about 100 days, about 50 days to about 120 days, or about 100 days to about 120 days. In some embodiments, the hydrogel fibers obtained by the method described herein comprise a flexible dissolution rate from about 1 day, about 2 days, about 3 days, about 4 days, about 5 days, about 8 days, about 10 days, about 20 days, about 30 days, about 50 days, about 100 days, or about 120 days. In some embodiments, the hydrogel fibers obtained by the method described herein comprise a flexible dissolution rate from at least about 1 day, about 2 days, about 3 days, about 4 days, about 5 days, about 8 days, about 10 days, about 20 days, about 30 days, about 50 days, or about 100 days. In some embodiments, the hydrogel fibers obtained by the method described herein comprise a flexible dissolution rate from at most about 2 days, about 3 days, about 4 days, about 5 days, about 8 days, about 10 days, about 20 days, about 30 days, about 50 days, about 100 days, or about 120 days.

In some embodiments, the hydrogel fibers obtained by the method described herein comprise a controllable gelation time from about 5 seconds to about 12 minutes. In some embodiments, the hydrogel fibers obtained by the method described herein comprise a controllable gelation time from about 1 second to about 120 seconds. In some embodiments, the hydrogel fibers obtained by the method described herein comprise a controllable gelation time from about 1 second to about 2 seconds, about 1 second to about 3 seconds, about 1 second to about 4 seconds, about 1 second to about 5 seconds, about 1 second to about 8 seconds, about 1 second to about 10 seconds, about 1 second to about 20 seconds, about 1 second to about 30 seconds, about 1 second to about 50 seconds, about 1 second to about 100 seconds, about 1 second to about 120 seconds, about 2 seconds to about 3 seconds, about 2 seconds to about 4 seconds, about 2 seconds to about 5 seconds, about 2 seconds to about 8 seconds, about 2 seconds to about 10 seconds, about 2 seconds to about 20 seconds, about 2 seconds to about 30 seconds, about 2 seconds to about 50 seconds, about 2 seconds to about 100 seconds, about 2 seconds to about 120 seconds, about 3 seconds to about 4 seconds, about 3 seconds to about 5 seconds, about 3 seconds to about 8 seconds, about 3 seconds to about 10 seconds, about 3 seconds to about 20 seconds, about 3 seconds to about 30 seconds, about 3 seconds to about 50 seconds, about 3 seconds to about 100 seconds, about 3 seconds to about 120 seconds, about 4 seconds to about 5 seconds, about 4 seconds to about 8 seconds, about 4 seconds to about 10 seconds, about 4 seconds to about 20 seconds, about 4 seconds to about 30 seconds, about 4 seconds to about 50 seconds, about 4 seconds to about 100 seconds, about 4 seconds to about 120 seconds, about 5 seconds to about 8 seconds, about 5 seconds to about 10 seconds, about 5 seconds to about 20 seconds, about 5 seconds to about 30 seconds, about 5 seconds to about 50 seconds, about 5 seconds to about 100 seconds, about 5 seconds to about 120 seconds, about 8 seconds to about 10 seconds, about 8 seconds to about 20 seconds, about 8 seconds to about 30 seconds, about 8 seconds to about 50 seconds, about 8 seconds to about 100 seconds, about 8 seconds to about 120 seconds, about 10 seconds to about 20 seconds, about 10 seconds to about 30 seconds, about 10 seconds to about 50 seconds, about 10 seconds to about 100 seconds, about 10 seconds to about 120 seconds, about 20 seconds to about 30 seconds, about 20 seconds to about 50 seconds, about 20 seconds to about 100 seconds, about 20 seconds to about 120 seconds, about 30 seconds to about 50 seconds, about 30 seconds to about 100 seconds, about 30 seconds to about 120 seconds, about 50 seconds to about 100 seconds, about 50 seconds to about 120 seconds, or about 100 seconds to about 120 seconds. In some embodiments, the hydrogel fibers obtained by the method described herein comprise a controllable gelation time from about 1 second, about 2 seconds, about 3 seconds, about 4 seconds, about 5 seconds, about 8 seconds, about 10 seconds, about 20 seconds, about 30 seconds, about 50 seconds, about 100 seconds, or about 120 seconds. In some embodiments, the hydrogel fibers obtained by the method described herein comprise a controllable gelation time from at least about 1 second, about 2 seconds, about 3 seconds, about 4 seconds, about 5 seconds, about 8 seconds, about 10 seconds, about 20 seconds, about 30 seconds, about 50 seconds, or about 100 seconds. In some embodiments, the hydrogel fibers obtained by the method described herein comprise a controllable gelation time from at most about 2 seconds, about 3 seconds, about 4 seconds, about 5 seconds, about 8 seconds, about 10 seconds, about 20 seconds, about 30 seconds, about 50 seconds, about 100 seconds, or about 120 seconds. In some embodiments, the hydrogel fibers obtained by the method described herein comprise a controllable gelation time from about 1 minute to about 100 minutes. In some embodiments, the hydrogel fibers obtained by the method described herein comprise a controllable gelation time from about 1 minute to about 2 minutes, about 1 minute to about 3 minutes, about 1 minute to about 4 minutes, about 1 minute to about 5 minutes, about 1 minute to about 8 minutes, about 1 minute to about 10 minutes, about 1 minute to about 12 minutes, about 1 minute to about 15 minutes, about 1 minute to about 20 minutes, about 1 minute to about 50 minutes, about 1 minute to about 100 minutes, about 2 minutes to about 3 minutes, about 2 minutes to about 4 minutes, about 2 minutes to about 5 minutes, about 2 minutes to about 8 minutes, about 2 minutes to about 10 minutes, about 2 minutes to about 12 minutes, about 2 minutes to about 15 minutes, about 2 minutes to about 20 minutes, about 2 minutes to about 50 minutes, about 2 minutes to about 100 minutes, about 3 minutes to about 4 minutes, about 3 minutes to about 5 minutes, about 3 minutes to about 8 minutes, about 3 minutes to about 10 minutes, about 3 minutes to about 12 minutes, about 3 minutes to about 15 minutes, about 3 minutes to about 20 minutes, about 3 minutes to about 50 minutes, about 3 minutes to about 100 minutes, about 4 minutes to about 5 minutes, about 4 minutes to about 8 minutes, about 4 minutes to about 10 minutes, about 4 minutes to about 12 minutes, about 4 minutes to about 15 minutes, about 4 minutes to about 20 minutes, about 4 minutes to about 50 minutes, about 4 minutes to about 100 minutes, about 5 minutes to about 8 minutes, about 5 minutes to about 10 minutes, about 5 minutes to about 12 minutes, about 5 minutes to about 15 minutes, about 5 minutes to about 20 minutes, about 5 minutes to about 50 minutes, about 5 minutes to about 100 minutes, about 8 minutes to about 10 minutes, about 8 minutes to about 12 minutes, about 8 minutes to about 15 minutes, about 8 minutes to about 20 minutes, about 8 minutes to about 50 minutes, about 8 minutes to about 100 minutes, about 10 minutes to about 12 minutes, about 10 minutes to about 15 minutes, about 10 minutes to about 20 minutes, about 10 minutes to about 50 minutes, about 10 minutes to about 100 minutes, about 12 minutes to about 15 minutes, about 12 minutes to about 20 minutes, about 12 minutes to about 50 minutes, about 12 minutes to about 100 minutes, about 15 minutes to about 20 minutes, about 15 minutes to about 50 minutes, about 15 minutes to about 100 minutes, about 20 minutes to about 50 minutes, about 20 minutes to about 100 minutes, or about 50 minutes to about 100 minutes. In some embodiments, the hydrogel fibers obtained by the method described herein comprise a controllable gelation time from about 1 minute, about 2 minutes, about 3 minutes, about 4 minutes, about 5 minutes, about 8 minutes, about 10 minutes, about 12 minutes, about 15 minutes, about 20 minutes, about 50 minutes, or about 100 minutes. In some embodiments, the hydrogel fibers obtained by the method described herein comprise a controllable gelation time from at least about 1 minute, about 2 minutes, about 3 minutes, about 4 minutes, about 5 minutes, about 8 minutes, about 10 minutes, about 12 minutes, about 15 minutes, about 20 minutes, or about 50 minutes. In some embodiments, the hydrogel fibers obtained by the method described herein comprise a controllable gelation time from at most about 2 minutes, about 3 minutes, about 4 minutes, about 5 minutes, about 8 minutes, about 10 minutes, about 12 minutes, about 15 minutes, about 20 minutes, about 50 minutes, or about 100 minutes.

In some embodiments, the hydrogel fibers obtained by the method described herein comprise an average width from about 20 micrometers to about 2,000 micrometers. In some embodiments, the hydrogel fibers obtained by the method described herein comprise an average width from about 20 micrometers to about 30 micrometers, about 20 micrometers to about 40 micrometers, about 20 micrometers to about 50 micrometers, about 20 micrometers to about 100 micrometers, about 20 micrometers to about 300 micrometers, about 20 micrometers to about 500 micrometers, about 20 micrometers to about 700 micrometers, about 20 micrometers to about 800 micrometers, about 20 micrometers to about 1,000 micrometers, about 20 micrometers to about 1,500 micrometers, about 20 micrometers to about 2,000 micrometers, about 30 micrometers to about 40 micrometers, about 30 micrometers to about 50 micrometers, about 30 micrometers to about 100 micrometers, about 30 micrometers to about 300 micrometers, about 30 micrometers to about 500 micrometers, about 30 micrometers to about 700 micrometers, about 30 micrometers to about 800 micrometers, about 30 micrometers to about 1,000 micrometers, about 30 micrometers to about 1,500 micrometers, about 30 micrometers to about 2,000 micrometers, about 40 micrometers to about 50 micrometers, about 40 micrometers to about 100 micrometers, about 40 micrometers to about 300 micrometers, about 40 micrometers to about 500 micrometers, about 40 micrometers to about 700 micrometers, about 40 micrometers to about 800 micrometers, about 40 micrometers to about 1,000 micrometers, about 40 micrometers to about 1,500 micrometers, about 40 micrometers to about 2,000 micrometers, about 50 micrometers to about 100 micrometers, about 50 micrometers to about 300 micrometers, about 50 micrometers to about 500 micrometers, about 50 micrometers to about 700 micrometers, about 50 micrometers to about 800 micrometers, about 50 micrometers to about 1,000 micrometers, about 50 micrometers to about 1,500 micrometers, about 50 micrometers to about 2,000 micrometers, about 100 micrometers to about 300 micrometers, about 100 micrometers to about 500 micrometers, about 100 micrometers to about 700 micrometers, about 100 micrometers to about 800 micrometers, about 100 micrometers to about 1,000 micrometers, about 100 micrometers to about 1,500 micrometers, about 100 micrometers to about 2,000 micrometers, about 300 micrometers to about 500 micrometers, about 300 micrometers to about 700 micrometers, about 300 micrometers to about 800 micrometers, about 300 micrometers to about 1,000 micrometers, about 300 micrometers to about 1,500 micrometers, about 300 micrometers to about 2,000 micrometers, about 500 micrometers to about 700 micrometers, about 500 micrometers to about 800 micrometers, about 500 micrometers to about 1,000 micrometers, about 500 micrometers to about 1,500 micrometers, about 500 micrometers to about 2,000 micrometers, about 700 micrometers to about 800 micrometers, about 700 micrometers to about 1,000 micrometers, about 700 micrometers to about 1,500 micrometers, about 700 micrometers to about 2,000 micrometers, about 800 micrometers to about 1,000 micrometers, about 800 micrometers to about 1,500 micrometers, about 800 micrometers to about 2,000 micrometers, about 1,000 micrometers to about 1,500 micrometers, about 1,000 micrometers to about 2,000 micrometers, or about 1,500 micrometers to about 2,000 micrometers. In some embodiments, the hydrogel fibers obtained by the method described herein comprise an average width from about 20 micrometers, about 30 micrometers, about 40 micrometers, about 50 micrometers, about 100 micrometers, about 300 micrometers, about 500 micrometers, about 700 micrometers, about 800 micrometers, about 1,000 micrometers, about 1,500 micrometers, or about 2,000 micrometers. In some embodiments, the hydrogel fibers obtained by the method described herein comprise an average width from at least about 20 micrometers, about 30 micrometers, about 40 micrometers, about 50 micrometers, about 100 micrometers, about 300 micrometers, about 500 micrometers, about 700 micrometers, about 800 micrometers, about 1,000 micrometers, or about 1,500 micrometers. In some embodiments, the hydrogel fibers obtained by the method described herein comprise an average width from at most about 30 micrometers, about 40 micrometers, about 50 micrometers, about 100 micrometers, about 300 micrometers, about 500 micrometers, about 700 micrometers, about 800 micrometers, about 1,000 micrometers, about 1,500 micrometers, or about 2,000 micrometers. In some embodiments, the hydrogel fibers obtained by the method described herein comprise an average width from about 20 millimeters to about 2,000 millimeters. In some embodiments, the hydrogel fibers obtained by the method described herein comprise an average width from about 20 millimeters to about 30 millimeters, about 20 millimeters to about 40 millimeters, about 20 millimeters to about 50 millimeters, about 20 millimeters to about 100 millimeters, about 20 millimeters to about 300 millimeters, about 20 millimeters to about 500 millimeters, about 20 millimeters to about 700 millimeters, about 20 millimeters to about 800 millimeters, about 20 millimeters to about 1,000 millimeters, about 20 millimeters to about 1,500 millimeters, about 20 millimeters to about 2,000 millimeters, about 30 millimeters to about 40 millimeters, about 30 millimeters to about 50 millimeters, about 30 millimeters to about 100 millimeters, about 30 millimeters to about 300 millimeters, about 30 millimeters to about 500 millimeters, about 30 millimeters to about 700 millimeters, about 30 millimeters to about 800 millimeters, about 30 millimeters to about 1,000 millimeters, about 30 millimeters to about 1,500 millimeters, about 30 millimeters to about 2,000 millimeters, about 40 millimeters to about 50 millimeters, about 40 millimeters to about 100 millimeters, about 40 millimeters to about 300 millimeters, about 40 millimeters to about 500 millimeters, about 40 millimeters to about 700 millimeters, about 40 millimeters to about 800 millimeters, about 40 millimeters to about 1,000 millimeters, about 40 millimeters to about 1,500 millimeters, about 40 millimeters to about 2,000 millimeters, about 50 millimeters to about 100 millimeters, about 50 millimeters to about 300 millimeters, about 50 millimeters to about 500 millimeters, about 50 millimeters to about 700 millimeters, about 50 millimeters to about 800 millimeters, about 50 millimeters to about 1,000 millimeters, about 50 millimeters to about 1,500 millimeters, about 50 millimeters to about 2,000 millimeters, about 100 millimeters to about 300 millimeters, about 100 millimeters to about 500 millimeters, about 100 millimeters to about 700 millimeters, about 100 millimeters to about 800 millimeters, about 100 millimeters to about 1,000 millimeters, about 100 millimeters to about 1,500 millimeters, about 100 millimeters to about 2,000 millimeters, about 300 millimeters to about 500 millimeters, about 300 millimeters to about 700 millimeters, about 300 millimeters to about 800 millimeters, about 300 millimeters to about 1,000 millimeters, about 300 millimeters to about 1,500 millimeters, about 300 millimeters to about 2,000 millimeters, about 500 millimeters to about 700 millimeters, about 500 millimeters to about 800 millimeters, about 500 millimeters to about 1,000 millimeters, about 500 millimeters to about 1,500 millimeters, about 500 millimeters to about 2,000 millimeters, about 700 millimeters to about 800 millimeters, about 700 millimeters to about 1,000 millimeters, about 700 millimeters to about 1,500 millimeters, about 700 millimeters to about 2,000 millimeters, about 800 millimeters to about 1,000 millimeters, about 800 millimeters to about 1,500 millimeters, about 800 millimeters to about 2,000 millimeters, about 1,000 millimeters to about 1,500 millimeters, about 1,000 millimeters to about 2,000 millimeters, or about 1,500 millimeters to about 2,000 millimeters. In some embodiments, the hydrogel fibers obtained by the method described herein comprise an average width from about 20 millimeters, about 30 millimeters, about 40 millimeters, about 50 millimeters, about 100 millimeters, about 300 millimeters, about 500 millimeters, about 700 millimeters, about 800 millimeters, about 1,000 millimeters, about 1,500 millimeters, or about 2,000 millimeters. In some embodiments, the hydrogel fibers obtained by the method described herein comprise an average width from at least about 20 millimeters, about 30 millimeters, about 40 millimeters, about 50 millimeters, about 100 millimeters, about 300 millimeters, about 500 millimeters, about 700 millimeters, about 800 millimeters, about 1,000 millimeters, or about 1,500 millimeters. In some embodiments, the hydrogel fibers obtained by the method described herein comprise an average width from at most about 30 millimeters, about 40 millimeters, about 50 millimeters, about 100 millimeters, about 300 millimeters, about 500 millimeters, about 700 millimeters, about 800 millimeters, about 1,000 millimeters, about 1,500 millimeters, or about 2,000 millimeters. In some embodiments, the hydrogel fibers obtained by the method described herein comprise an average width from about 1 centimeters to about 20 centimeters. In some embodiments, the hydrogel fibers obtained by the method described herein comprise an average width from about 1 centimeters to about 2 centimeters, about 1 centimeters to about 3 centimeters, about 1 centimeters to about 5 centimeters, about 1 centimeters to about 10 centimeters, about 1 centimeters to about 12 centimeters, about 1 centimeters to about 15 centimeters, about 1 centimeters to about 20 centimeters, about 2 centimeters to about 3 centimeters, about 2 centimeters to about 5 centimeters, about 2 centimeters to about 10 centimeters, about 2 centimeters to about 12 centimeters, about 2 centimeters to about 15 centimeters, about 2 centimeters to about 20 centimeters, about 3 centimeters to about 5 centimeters, about 3 centimeters to about 10 centimeters, about 3 centimeters to about 12 centimeters, about 3 centimeters to about 15 centimeters, about 3 centimeters to about 20 centimeters, about 5 centimeters to about 10 centimeters, about 5 centimeters to about 12 centimeters, about 5 centimeters to about 15 centimeters, about 5 centimeters to about 20 centimeters, about 10 centimeters to about 12 centimeters, about 10 centimeters to about 15 centimeters, about 10 centimeters to about 20 centimeters, about 12 centimeters to about 15 centimeters, about 12 centimeters to about 20 centimeters, or about 15 centimeters to about 20 centimeters. In some embodiments, the hydrogel fibers obtained by the method described herein comprise an average width from about 1 centimeters, about 2 centimeters, about 3 centimeters, about 5 centimeters, about 10 centimeters, about 12 centimeters, about 15 centimeters, or about 20 centimeters. In some embodiments, the hydrogel fibers obtained by the method described herein comprise an average width from at least about 1 centimeters, about 2 centimeters, about 3 centimeters, about 5 centimeters, about 10 centimeters, about 12 centimeters, or about 15 centimeters. In some embodiments, the hydrogel fibers obtained by the method described herein comprise an average width from at most about 2 centimeters, about 3 centimeters, about 5 centimeters, about 10 centimeters, about 12 centimeters, about 15 centimeters, or about 20 centimeters.

In some embodiments, the hydrogel fibers obtained by the method described herein comprise an average length from about 20 micrometers to about 2,000 micrometers. In some embodiments, the hydrogel fibers obtained by the method described herein comprise an average length from about 20 micrometers to about 30 micrometers, about 20 micrometers to about 40 micrometers, about 20 micrometers to about 50 micrometers, about 20 micrometers to about 100 micrometers, about 20 micrometers to about 300 micrometers, about 20 micrometers to about 500 micrometers, about 20 micrometers to about 700 micrometers, about 20 micrometers to about 800 micrometers, about 20 micrometers to about 1,000 micrometers, about 20 micrometers to about 1,500 micrometers, about 20 micrometers to about 2,000 micrometers, about 30 micrometers to about 40 micrometers, about 30 micrometers to about 50 micrometers, about 30 micrometers to about 100 micrometers, about 30 micrometers to about 300 micrometers, about 30 micrometers to about 500 micrometers, about 30 micrometers to about 700 micrometers, about 30 micrometers to about 800 micrometers, about 30 micrometers to about 1,000 micrometers, about 30 micrometers to about 1,500 micrometers, about 30 micrometers to about 2,000 micrometers, about 40 micrometers to about 50 micrometers, about 40 micrometers to about 100 micrometers, about 40 micrometers to about 300 micrometers, about 40 micrometers to about 500 micrometers, about 40 micrometers to about 700 micrometers, about 40 micrometers to about 800 micrometers, about 40 micrometers to about 1,000 micrometers, about 40 micrometers to about 1,500 micrometers, about 40 micrometers to about 2,000 micrometers, about 50 micrometers to about 100 micrometers, about 50 micrometers to about 300 micrometers, about 50 micrometers to about 500 micrometers, about 50 micrometers to about 700 micrometers, about 50 micrometers to about 800 micrometers, about 50 micrometers to about 1,000 micrometers, about 50 micrometers to about 1,500 micrometers, about 50 micrometers to about 2,000 micrometers, about 100 micrometers to about 300 micrometers, about 100 micrometers to about 500 micrometers, about 100 micrometers to about 700 micrometers, about 100 micrometers to about 800 micrometers, about 100 micrometers to about 1,000 micrometers, about 100 micrometers to about 1,500 micrometers, about 100 micrometers to about 2,000 micrometers, about 300 micrometers to about 500 micrometers, about 300 micrometers to about 700 micrometers, about 300 micrometers to about 800 micrometers, about 300 micrometers to about 1,000 micrometers, about 300 micrometers to about 1,500 micrometers, about 300 micrometers to about 2,000 micrometers, about 500 micrometers to about 700 micrometers, about 500 micrometers to about 800 micrometers, about 500 micrometers to about 1,000 micrometers, about 500 micrometers to about 1,500 micrometers, about 500 micrometers to about 2,000 micrometers, about 700 micrometers to about 800 micrometers, about 700 micrometers to about 1,000 micrometers, about 700 micrometers to about 1,500 micrometers, about 700 micrometers to about 2,000 micrometers, about 800 micrometers to about 1,000 micrometers, about 800 micrometers to about 1,500 micrometers, about 800 micrometers to about 2,000 micrometers, about 1,000 micrometers to about 1,500 micrometers, about 1,000 micrometers to about 2,000 micrometers, or about 1,500 micrometers to about 2,000 micrometers. In some embodiments, the hydrogel fibers obtained by the method described herein comprise an average length from about 20 micrometers, about 30 micrometers, about 40 micrometers, about 50 micrometers, about 100 micrometers, about 300 micrometers, about 500 micrometers, about 700 micrometers, about 800 micrometers, about 1,000 micrometers, about 1,500 micrometers, or about 2,000 micrometers. In some embodiments, the hydrogel fibers obtained by the method described herein comprise an average length from at least about 20 micrometers, about 30 micrometers, about 40 micrometers, about 50 micrometers, about 100 micrometers, about 300 micrometers, about 500 micrometers, about 700 micrometers, about 800 micrometers, about 1,000 micrometers, or about 1,500 micrometers. In some embodiments, the hydrogel fibers obtained by the method described herein comprise an average length from at most about 30 micrometers, about 40 micrometers, about 50 micrometers, about 100 micrometers, about 300 micrometers, about 500 micrometers, about 700 micrometers, about 800 micrometers, about 1,000 micrometers, about 1,500 micrometers, or about 2,000 micrometers. In some embodiments, the hydrogel fibers obtained by the method described herein comprise an average length from about 20 millimeters to about 2,000 millimeters. In some embodiments, the hydrogel fibers obtained by the method described herein comprise an average length from about 20 millimeters to about 30 millimeters, about 20 millimeters to about 40 millimeters, about 20 millimeters to about 50 millimeters, about 20 millimeters to about 100 millimeters, about 20 millimeters to about 300 millimeters, about 20 millimeters to about 500 millimeters, about 20 millimeters to about 700 millimeters, about 20 millimeters to about 800 millimeters, about 20 millimeters to about 1,000 millimeters, about 20 millimeters to about 1,500 millimeters, about 20 millimeters to about 2,000 millimeters, about 30 millimeters to about 40 millimeters, about 30 millimeters to about 50 millimeters, about 30 millimeters to about 100 millimeters, about 30 millimeters to about 300 millimeters, about 30 millimeters to about 500 millimeters, about 30 millimeters to about 700 millimeters, about 30 millimeters to about 800 millimeters, about 30 millimeters to about 1,000 millimeters, about 30 millimeters to about 1,500 millimeters, about 30 millimeters to about 2,000 millimeters, about 40 millimeters to about 50 millimeters, about 40 millimeters to about 100 millimeters, about 40 millimeters to about 300 millimeters, about 40 millimeters to about 500 millimeters, about 40 millimeters to about 700 millimeters, about 40 millimeters to about 800 millimeters, about 40 millimeters to about 1,000 millimeters, about 40 millimeters to about 1,500 millimeters, about 40 millimeters to about 2,000 millimeters, about 50 millimeters to about 100 millimeters, about 50 millimeters to about 300 millimeters, about 50 millimeters to about 500 millimeters, about 50 millimeters to about 700 millimeters, about 50 millimeters to about 800 millimeters, about 50 millimeters to about 1,000 millimeters, about 50 millimeters to about 1,500 millimeters, about 50 millimeters to about 2,000 millimeters, about 100 millimeters to about 300 millimeters, about 100 millimeters to about 500 millimeters, about 100 millimeters to about 700 millimeters, about 100 millimeters to about 800 millimeters, about 100 millimeters to about 1,000 millimeters, about 100 millimeters to about 1,500 millimeters, about 100 millimeters to about 2,000 millimeters, about 300 millimeters to about 500 millimeters, about 300 millimeters to about 700 millimeters, about 300 millimeters to about 800 millimeters, about 300 millimeters to about 1,000 millimeters, about 300 millimeters to about 1,500 millimeters, about 300 millimeters to about 2,000 millimeters, about 500 millimeters to about 700 millimeters, about 500 millimeters to about 800 millimeters, about 500 millimeters to about 1,000 millimeters, about 500 millimeters to about 1,500 millimeters, about 500 millimeters to about 2,000 millimeters, about 700 millimeters to about 800 millimeters, about 700 millimeters to about 1,000 millimeters, about 700 millimeters to about 1,500 millimeters, about 700 millimeters to about 2,000 millimeters, about 800 millimeters to about 1,000 millimeters, about 800 millimeters to about 1,500 millimeters, about 800 millimeters to about 2,000 millimeters, about 1,000 millimeters to about 1,500 millimeters, about 1,000 millimeters to about 2,000 millimeters, or about 1,500 millimeters to about 2,000 millimeters. In some embodiments, the hydrogel fibers obtained by the method described herein comprise an average length from about 20 millimeters, about 30 millimeters, about 40 millimeters, about 50 millimeters, about 100 millimeters, about 300 millimeters, about 500 millimeters, about 700 millimeters, about 800 millimeters, about 1,000 millimeters, about 1,500 millimeters, or about 2,000 millimeters. In some embodiments, the hydrogel fibers obtained by the method described herein comprise an average length from at least about 20 millimeters, about 30 millimeters, about 40 millimeters, about 50 millimeters, about 100 millimeters, about 300 millimeters, about 500 millimeters, about 700 millimeters, about 800 millimeters, about 1,000 millimeters, or about 1,500 millimeters. In some embodiments, the hydrogel fibers obtained by the method described herein comprise an average length from at most about 30 millimeters, about 40 millimeters, about 50 millimeters, about 100 millimeters, about 300 millimeters, about 500 millimeters, about 700 millimeters, about 800 millimeters, about 1,000 millimeters, about 1,500 millimeters, or about 2,000 millimeters. In some embodiments, the hydrogel fibers obtained by the method described herein comprise an average length from about 1 centimeters to about 20 centimeters. In some embodiments, the hydrogel fibers obtained by the method described herein comprise an average length from about 1 centimeters to about 2 centimeters, about 1 centimeters to about 3 centimeters, about 1 centimeters to about 5 centimeters, about 1 centimeters to about 10 centimeters, about 1 centimeters to about 12 centimeters, about 1 centimeters to about 15 centimeters, about 1 centimeters to about 20 centimeters, about 2 centimeters to about 3 centimeters, about 2 centimeters to about 5 centimeters, about 2 centimeters to about 10 centimeters, about 2 centimeters to about 12 centimeters, about 2 centimeters to about 15 centimeters, about 2 centimeters to about 20 centimeters, about 3 centimeters to about 5 centimeters, about 3 centimeters to about 10 centimeters, about 3 centimeters to about 12 centimeters, about 3 centimeters to about 15 centimeters, about 3 centimeters to about 20 centimeters, about 5 centimeters to about 10 centimeters, about 5 centimeters to about 12 centimeters, about 5 centimeters to about 15 centimeters, about 5 centimeters to about 20 centimeters, about 10 centimeters to about 12 centimeters, about 10 centimeters to about 15 centimeters, about 10 centimeters to about 20 centimeters, about 12 centimeters to about 15 centimeters, about 12 centimeters to about 20 centimeters, or about 15 centimeters to about 20 centimeters. In some embodiments, the hydrogel fibers obtained by the method described herein comprise an average length from about 1 centimeters, about 2 centimeters, about 3 centimeters, about 5 centimeters, about 10 centimeters, about 12 centimeters, about 15 centimeters, or about 20 centimeters. In some embodiments, the hydrogel fibers obtained by the method described herein comprise an average length from at least about 1 centimeters, about 2 centimeters, about 3 centimeters, about 5 centimeters, about 10 centimeters, about 12 centimeters, or about 15 centimeters. In some embodiments, the hydrogel fibers obtained by the method described herein comprise an average length from at most about 2 centimeters, about 3 centimeters, about 5 centimeters, about 10 centimeters, about 12 centimeters, about 15 centimeters, or about 20 centimeters.

In some embodiments, the hydrogel fibers obtained by the method described herein comprise an average density of about 10 mole per cubic meter to about 65 mole per cubic meter. In some embodiments, the hydrogel fibers obtained by the method described herein comprise an average density of about 10 mole per cubic meter to about 15 mole per cubic meter, about 10 mole per cubic meter to about 19 mole per cubic meter, about 10 mole per cubic meter to about 25 mole per cubic meter, about 10 mole per cubic meter to about 30 mole per cubic meter, about 10 mole per cubic meter to about 35 mole per cubic meter, about 10 mole per cubic meter to about 40 mole per cubic meter, about 10 mole per cubic meter to about 45 mole per cubic meter, about 10 mole per cubic meter to about 50 mole per cubic meter, about 10 mole per cubic meter to about 56 mole per cubic meter, about 10 mole per cubic meter to about 60 mole per cubic meter, about 10 mole per cubic meter to about 65 mole per cubic meter, about 15 mole per cubic meter to about 19 mole per cubic meter, about 15 mole per cubic meter to about 25 mole per cubic meter, about 15 mole per cubic meter to about 30 mole per cubic meter, about 15 mole per cubic meter to about 35 mole per cubic meter, about 15 mole per cubic meter to about 40 mole per cubic meter, about 15 mole per cubic meter to about 45 mole per cubic meter, about 15 mole per cubic meter to about 50 mole per cubic meter, about 15 mole per cubic meter to about 56 mole per cubic meter, about 15 mole per cubic meter to about 60 mole per cubic meter, about 15 mole per cubic meter to about 65 mole per cubic meter, about 19 mole per cubic meter to about 25 mole per cubic meter, about 19 mole per cubic meter to about 30 mole per cubic meter, about 19 mole per cubic meter to about 35 mole per cubic meter, about 19 mole per cubic meter to about 40 mole per cubic meter, about 19 mole per cubic meter to about 45 mole per cubic meter, about 19 mole per cubic meter to about 50 mole per cubic meter, about 19 mole per cubic meter to about 56 mole per cubic meter, about 19 mole per cubic meter to about 60 mole per cubic meter, about 19 mole per cubic meter to about 65 mole per cubic meter, about 25 mole per cubic meter to about 30 mole per cubic meter, about 25 mole per cubic meter to about 35 mole per cubic meter, about 25 mole per cubic meter to about 40 mole per cubic meter, about 25 mole per cubic meter to about 45 mole per cubic meter, about 25 mole per cubic meter to about 50 mole per cubic meter, about 25 mole per cubic meter to about 56 mole per cubic meter, about 25 mole per cubic meter to about 60 mole per cubic meter, about 25 mole per cubic meter to about 65 mole per cubic meter, about 30 mole per cubic meter to about 35 mole per cubic meter, about 30 mole per cubic meter to about 40 mole per cubic meter, about 30 mole per cubic meter to about 45 mole per cubic meter, about 30 mole per cubic meter to about 50 mole per cubic meter, about 30 mole per cubic meter to about 56 mole per cubic meter, about 30 mole per cubic meter to about 60 mole per cubic meter, about 30 mole per cubic meter to about 65 mole per cubic meter, about 35 mole per cubic meter to about 40 mole per cubic meter, about 35 mole per cubic meter to about 45 mole per cubic meter, about 35 mole per cubic meter to about 50 mole per cubic meter, about 35 mole per cubic meter to about 56 mole per cubic meter, about 35 mole per cubic meter to about 60 mole per cubic meter, about 35 mole per cubic meter to about 65 mole per cubic meter, about 40 mole per cubic meter to about 45 mole per cubic meter, about 40 mole per cubic meter to about 50 mole per cubic meter, about 40 mole per cubic meter to about 56 mole per cubic meter, about 40 mole per cubic meter to about 60 mole per cubic meter, about 40 mole per cubic meter to about 65 mole per cubic meter, about 45 mole per cubic meter to about 50 mole per cubic meter, about 45 mole per cubic meter to about 56 mole per cubic meter, about 45 mole per cubic meter to about 60 mole per cubic meter, about 45 mole per cubic meter to about 65 mole per cubic meter, about 50 mole per cubic meter to about 56 mole per cubic meter, about 50 mole per cubic meter to about 60 mole per cubic meter, about 50 mole per cubic meter to about 65 mole per cubic meter, about 56 mole per cubic meter to about 60 mole per cubic meter, about 56 mole per cubic meter to about 65 mole per cubic meter, or about 60 mole per cubic meter to about 65 mole per cubic meter. In some embodiments, the hydrogel fibers obtained by the method described herein comprise an average density of about 10 mole per cubic meter, about 15 mole per cubic meter, about 19 mole per cubic meter, about 25 mole per cubic meter, about 30 mole per cubic meter, about 35 mole per cubic meter, about 40 mole per cubic meter, about 45 mole per cubic meter, about 50 mole per cubic meter, about 56 mole per cubic meter, about 60 mole per cubic meter, or about 65 mole per cubic meter. In some embodiments, the hydrogel fibers obtained by the method described herein comprise an average density of at least about 10 mole per cubic meter, about 15 mole per cubic meter, about 19 mole per cubic meter, about 25 mole per cubic meter, about 30 mole per cubic meter, about 35 mole per cubic meter, about 40 mole per cubic meter, about 45 mole per cubic meter, about 50 mole per cubic meter, about 56 mole per cubic meter, or about 60 mole per cubic meter. In some embodiments, the hydrogel fibers obtained by the method described herein comprise an average density of at most about 15 mole per cubic meter, about 19 mole per cubic meter, about 25 mole per cubic meter, about 30 mole per cubic meter, about 35 mole per cubic meter, about 40 mole per cubic meter, about 45 mole per cubic meter, about 50 mole per cubic meter, about 56 mole per cubic meter, about 60 mole per cubic meter, or about 65 mole per cubic meter.

In some embodiments, the hydrogel fibers obtained by the method described herein comprise a low-rigidity elasticity about 1 kilopascal to about 40 kilopascals. In some embodiments, the hydrogel fibers obtained by the method described herein comprise a low-rigidity elasticity about 1 kilopascal to about 2 kilopascals, about 1 kilopascal to about 3 kilopascals, about 1 kilopascal to about 5 kilopascals, about 1 kilopascal to about 10 kilopascals, about 1 kilopascal to about 15 kilopascals, about 1 kilopascal to about 20 kilopascals, about 1 kilopascal to about 25 kilopascals, about 1 kilopascal to about 30 kilopascals, about 1 kilopascal to about 35 kilopascals, about 1 kilopascal to about 40 kilopascals, about 2 kilopascals to about 3 kilopascals, about 2 kilopascals to about 5 kilopascals, about 2 kilopascals to about 10 kilopascals, about 2 kilopascals to about 15 kilopascals, about 2 kilopascals to about 20 kilopascals, about 2 kilopascals to about 25 kilopascals, about 2 kilopascals to about 30 kilopascals, about 2 kilopascals to about 35 kilopascals, about 2 kilopascals to about 40 kilopascals, about 3 kilopascals to about 5 kilopascals, about 3 kilopascals to about 10 kilopascals, about 3 kilopascals to about 15 kilopascals, about 3 kilopascals to about 20 kilopascals, about 3 kilopascals to about 25 kilopascals, about 3 kilopascals to about 30 kilopascals, about 3 kilopascals to about 35 kilopascals, about 3 kilopascals to about 40 kilopascals, about 5 kilopascals to about 10 kilopascals, about 5 kilopascals to about 15 kilopascals, about 5 kilopascals to about 20 kilopascals, about 5 kilopascals to about 25 kilopascals, about 5 kilopascals to about 30 kilopascals, about 5 kilopascals to about 35 kilopascals, about 5 kilopascals to about 40 kilopascals, about 10 kilopascals to about 15 kilopascals, about 10 kilopascals to about 20 kilopascals, about 10 kilopascals to about 25 kilopascals, about 10 kilopascals to about 30 kilopascals, about 10 kilopascals to about 35 kilopascals, about 10 kilopascals to about 40 kilopascals, about 15 kilopascals to about 20 kilopascals, about 15 kilopascals to about 25 kilopascals, about 15 kilopascals to about 30 kilopascals, about 15 kilopascals to about 35 kilopascals, about 15 kilopascals to about 40 kilopascals, about 20 kilopascals to about 25 kilopascals, about 20 kilopascals to about 30 kilopascals, about 20 kilopascals to about 35 kilopascals, about 20 kilopascals to about 40 kilopascals, about 25 kilopascals to about 30 kilopascals, about 25 kilopascals to about 35 kilopascals, about 25 kilopascals to about 40 kilopascals, about 30 kilopascals to about 35 kilopascals, about 30 kilopascals to about 40 kilopascals, or about 35 kilopascals to about 40 kilopascals. In some embodiments, the hydrogel fibers obtained by the method described herein comprise a low-rigidity elasticity about 1 kilopascal, about 2 kilopascals, about 3 kilopascals, about 5 kilopascals, about 10 kilopascals, about 15 kilopascals, about 20 kilopascals, about 25 kilopascals, about 30 kilopascals, about 35 kilopascals, or about 40 kilopascals. In some embodiments, the hydrogel fibers obtained by the method described herein comprise a low-rigidity elasticity at least about 1 kilopascal, about 2 kilopascals, about 3 kilopascals, about 5 kilopascals, about 10 kilopascals, about 15 kilopascals, about 20 kilopascals, about 25 kilopascals, about 30 kilopascals, or about 35 kilopascals. In some embodiments, the hydrogel fibers obtained by the method described herein comprise a low-rigidity elasticity at most about 2 kilopascals, about 3 kilopascals, about 5 kilopascals, about 10 kilopascals, about 15 kilopascals, about 20 kilopascals, about 25 kilopascals, about 30 kilopascals, about 35 kilopascals, or about 40 kilopascals.

In some embodiments, the hydrogel fibers obtained by the method described herein comprise a porous surface with an average pore size opening possessing a width about 1 micrometer to about 3,000 micrometers. In some embodiments, the hydrogel fibers obtained by the method described herein comprise a porous surface with an average pore size opening possessing a width about 1 micrometer to about 2 micrometers, about 1 micrometer to about 3 micrometers, about 1 micrometer to about 5 micrometers, about 1 micrometer to about 10 micrometers, about 1 micrometer to about 50 micrometers, about 1 micrometer to about 100 micrometers, about 1 micrometer to about 200 micrometers, about 1 micrometer to about 3,000 micrometers, about 1 micrometer to about 400 micrometers, about 1 micrometer to about 500 micrometers, about 1 micrometer to about 1,000 micrometers, about 2 micrometers to about 3 micrometers, about 2 micrometers to about 5 micrometers, about 2 micrometers to about 10 micrometers, about 2 micrometers to about 50 micrometers, about 2 micrometers to about 100 micrometers, about 2 micrometers to about 200 micrometers, about 2 micrometers to about 3,000 micrometers, about 2 micrometers to about 400 micrometers, about 2 micrometers to about 500 micrometers, about 2 micrometers to about 1,000 micrometers, about 3 micrometers to about 5 micrometers, about 3 micrometers to about 10 micrometers, about 3 micrometers to about 50 micrometers, about 3 micrometers to about 100 micrometers, about 3 micrometers to about 200 micrometers, about 3 micrometers to about 3,000 micrometers, about 3 micrometers to about 400 micrometers, about 3 micrometers to about 500 micrometers, about 3 micrometers to about 1,000 micrometers, about 5 micrometers to about 10 micrometers, about 5 micrometers to about 50 micrometers, about 5 micrometers to about 100 micrometers, about 5 micrometers to about 200 micrometers, about 5 micrometers to about 3,000 micrometers, about 5 micrometers to about 400 micrometers, about 5 micrometers to about 500 micrometers, about 5 micrometers to about 1,000 micrometers, about 10 micrometers to about 50 micrometers, about 10 micrometers to about 100 micrometers, about 10 micrometers to about 200 micrometers, about 10 micrometers to about 3,000 micrometers, about 10 micrometers to about 400 micrometers, about 10 micrometers to about 500 micrometers, about 10 micrometers to about 1,000 micrometers, about 50 micrometers to about 100 micrometers, about 50 micrometers to about 200 micrometers, about 50 micrometers to about 3,000 micrometers, about 50 micrometers to about 400 micrometers, about 50 micrometers to about 500 micrometers, about 50 micrometers to about 1,000 micrometers, about 100 micrometers to about 200 micrometers, about 100 micrometers to about 3,000 micrometers, about 100 micrometers to about 400 micrometers, about 100 micrometers to about 500 micrometers, about 100 micrometers to about 1,000 micrometers, about 200 micrometers to about 3,000 micrometers, about 200 micrometers to about 400 micrometers, about 200 micrometers to about 500 micrometers, about 200 micrometers to about 1,000 micrometers, about 3,000 micrometers to about 400 micrometers, about 3,000 micrometers to about 500 micrometers, about 3,000 micrometers to about 1,000 micrometers, about 400 micrometers to about 500 micrometers, about 400 micrometers to about 1,000 micrometers, or about 500 micrometers to about 1,000 micrometers. In some embodiments, the hydrogel fibers obtained by the method described herein comprise a porous surface with an average pore size opening possessing a width about 1 micrometer, about 2 micrometers, about 3 micrometers, about 5 micrometers, about 10 micrometers, about 50 micrometers, about 100 micrometers, about 200 micrometers, about 3,000 micrometers, about 400 micrometers, about 500 micrometers, or about 1,000 micrometers. In some embodiments, the hydrogel fibers obtained by the method described herein comprise a porous surface with an average pore size opening possessing a width at least about 1 micrometer, about 2 micrometers, about 3 micrometers, about 5 micrometers, about 10 micrometers, about 50 micrometers, about 100 micrometers, about 200 micrometers, about 3,000 micrometers, about 400 micrometers, or about 500 micrometers. In some embodiments, the hydrogel fibers obtained by the method described herein comprise a porous surface with an average pore size opening possessing a width at most about 2 micrometers, about 3 micrometers, about 5 micrometers, about 10 micrometers, about 50 micrometers, about 100 micrometers, about 200 micrometers, about 3,000 micrometers, about 400 micrometers, about 500 micrometers, or about 1,000 micrometers.

In some embodiments, the hydrogel fibers are water-stable. In some embodiments, the hydrogel fibers are bio-compatible. In some embodiments, the hydrogel fibers comprise a thermoreversible hydrogel that is not a liquid at room temperature. In some embodiments, the hydrogel fibers comprise a thermoreversible hydrogel with a gelation temperature (Tgel) from about 1° C. to about 70° C. In some embodiments, the hydrogel fibers comprise a thermoreversible hydrogel with a gelation temperature (Tgel) from about 1° C. to about 2° C., about 1° C. to about 3° C., about 1° C. to about 5° C., about 1° C. to about 10° C., about 1° C. to about 20° C., about 1° C. to about 30° C., about 1° C. to about 40° C., about 1° C. to about 50° C., about 1° C. to about 60° C., about 1° C. to about 70° C., about 2° C. to about 3° C., about 2° C. to about 5° C., about 2° C. to about 10° C., about 2° C. to about 20° C., about 2° C. to about 30° C., about 2° C. to about 40° C., about 2° C. to about 50° C., about 2° C. to about 60° C., about 2° C. to about 70° C., about 3° C. to about 5° C., about 3° C. to about 10° C., about 3° C. to about 20° C., about 3° C. to about 30° C., about 3° C. to about 40° C., about 3° C. to about 50° C., about 3° C. to about 60° C., about 3° C. to about 70° C., about 5° C. to about 10° C., about 5° C. to about 20° C., about 5° C. to about 30° C., about 5° C. to about 40° C., about 5° C. to about 50° C., about 5° C. to about 60° C., about 5° C. to about 70° C., about 10° C. to about 20° C., about 10° C. to about 30° C., about 10° C. to about 40° C., about 10° C. to about 50° C., about 10° C. to about 60° C., about 10° C. to about 70° C., about 20° C. to about 30° C., about 20° C. to about 40° C., about 20° C. to about 50° C., about 20° C. to about 60° C., about 20° C. to about 70° C., about 30° C. to about 40° C., about 30° C. to about 50° C., about 30° C. to about 60° C., about 30° C. to about 70° C., about 40° C. to about 50° C., about 40° C. to about 60° C., about 40° C. to about 70° C., about 50° C. to about 60° C., about 50° C. to about 70° C., or about 60° C. to about 70° C. In some embodiments, the hydrogel fibers comprise a thermoreversible hydrogel with a gelation temperature (Tgel) from about 1° C., about 2° C., about 3° C., about 5° C., about 10° C., about 20° C., about 30° C., about 40° C., about 50° C., about 60° C., or about 70° C. In some embodiments, the hydrogel fibers comprise a thermoreversible hydrogel with a gelation temperature (Tgel) from at least about 1° C., about 2° C., about 3° C., about 5° C., about 10° C., about 20° C., about 30° C., about 40° C., about 50° C., or about 60° C. In some embodiments, the hydrogel fibers comprise a thermoreversible hydrogel with a gelation temperature (Tgel) from at most about 2° C., about 3° C., about 5° C., about 10° C., about 20° C., about 30° C., about 40° C., about 50° C., about 60° C., or about 70° C.

In some embodiments, the method comprises contacting the scaffold comprising the crosslinked hydrogel fibers describe herein with cells or cell precursors from a non-human animal source. In some embodiments, the cells or cell precursors from a non-human animal source comprise cells from a tissue biopsy, an immortalized cell line, blood, stem cells, precursor cells, embryonic cells, bone marrow, or any combination thereof. Non-human animal source can include cow, a pig, a chicken, a fish, a sheep, a bison, a duck, a goose, an elk, a deer, a Berkshire pig, a Kurobuta pig, an Iberian pig, an ostrich, and combinations thereof. In some embodiments, the method comprises screening cells or cultured fibers for metabolic activity. In some embodiments, the method comprises expanding the cells or cell precursors for 12 hours to 10 days to obtain cell cultured fibers. In some embodiments, the method includes expanding the cells or cell precursors for 12 hours to 10 days to obtain cell cultured fibers in suspension culture. In some embodiments, the method comprises expanding the cells or cell precursors for 12 hours about 1 hour to about 72 hours. In some embodiments, the method comprises expanding the cells or cell precursors for 12 hours about 1 hour to about 2 hours, about 1 hour to about 3 hours, about 1 hour to about 5 hours, about 1 hour to about 10 hours, about 1 hour to about 12 hours, about 1 hour to about 24 hours, about 1 hour to about 36 hours, about 1 hour to about 48 hours, about 1 hour to about 72 hours, about 2 hours to about 3 hours, about 2 hours to about 5 hours, about 2 hours to about 10 hours, about 2 hours to about 12 hours, about 2 hours to about 24 hours, about 2 hours to about 36 hours, about 2 hours to about 48 hours, about 2 hours to about 72 hours, about 3 hours to about 5 hours, about 3 hours to about 10 hours, about 3 hours to about 12 hours, about 3 hours to about 24 hours, about 3 hours to about 36 hours, about 3 hours to about 48 hours, about 3 hours to about 72 hours, about 5 hours to about 10 hours, about 5 hours to about 12 hours, about 5 hours to about 24 hours, about 5 hours to about 36 hours, about 5 hours to about 48 hours, about 5 hours to about 72 hours, about 10 hours to about 12 hours, about 10 hours to about 24 hours, about 10 hours to about 36 hours, about 10 hours to about 48 hours, about 10 hours to about 72 hours, about 12 hours to about 24 hours, about 12 hours to about 36 hours, about 12 hours to about 48 hours, about 12 hours to about 72 hours, about 24 hours to about 36 hours, about 24 hours to about 48 hours, about 24 hours to about 72 hours, about 36 hours to about 48 hours, about 36 hours to about 72 hours, or about 48 hours to about 72 hours. In some embodiments, the method comprises expanding the cells or cell precursors for 12 hours about 1 hour, about 2 hours, about 3 hours, about 5 hours, about 10 hours, about 12 hours, about 24 hours, about 36 hours, about 48 hours, or about 72 hours. In some embodiments, the method comprises expanding the cells or cell precursors for 12 hours at least about 1 hour, about 2 hours, about 3 hours, about 5 hours, about 10 hours, about 12 hours, about 24 hours, about 36 hours, or about 48 hours. In some embodiments, the method comprises expanding the cells or cell precursors for 12 hours at most about 2 hours, about 3 hours, about 5 hours, about 10 hours, about 12 hours, about 24 hours, about 36 hours, about 48 hours, or about 72 hours. In some embodiments, the method comprises expanding the cells or cell precursors for 12 hours about 1 day to about 30 days. In some embodiments, the method comprises expanding the cells or cell precursors for 12 hours about 1 day to about 2 days, about 1 day to about 3 days, about 1 day to about 5 days, about 1 day to about 10 days, about 1 day to about 30 days, about 2 days to about 3 days, about 2 days to about 5 days, about 2 days to about 10 days, about 2 days to about 30 days, about 3 days to about 5 days, about 3 days to about 10 days, about 3 days to about 30 days, about 5 days to about 10 days, about 5 days to about 30 days, or about 10 days to about 30 days. In some embodiments, the method comprises expanding the cells or cell precursors for 12 hours about 1 day, about 2 days, about 3 days, about 5 days, about 10 days, or about 30 days. In some embodiments, the method comprises expanding the cells or cell precursors for 12 hours at least about 1 day, about 2 days, about 3 days, about 5 days, or about 10 days. In some embodiments, the method comprises expanding the cells or cell precursors for 12 hours at most about 2 days, about 3 days, about 5 days, about 10 days, or about 30 days.

In some embodiments, the method comprises harvesting the cell cultured fibers when the average width of the cell cultured fibers is from In some embodiments, the method comprises harvesting the cell cultured fibers when the average width of the cell cultured fibers is from about 10 micrometers to about 5,000 micrometers. In some embodiments, the method comprises harvesting the cell cultured fibers when the average width of the cell cultured fibers is from about 10 micrometers to about 20 micrometers, about 10 micrometers to about 30 micrometers, about 10 micrometers to about 40 micrometers, about 10 micrometers to about 100 micrometers, about 10 micrometers to about 200 micrometers, about 10 micrometers to about 500 micrometers, about 10 micrometers to about 1,000 micrometers, about 10 micrometers to about 2,000 micrometers, about 10 micrometers to about 3,000 micrometers, about 10 micrometers to about 5,000 micrometers, about 20 micrometers to about 30 micrometers, about 20 micrometers to about 40 micrometers, about 20 micrometers to about 100 micrometers, about 20 micrometers to about 200 micrometers, about 20 micrometers to about 500 micrometers, about 20 micrometers to about 1,000 micrometers, about 20 micrometers to about 2,000 micrometers, about 20 micrometers to about 3,000 micrometers, about 20 micrometers to about 5,000 micrometers, about 30 micrometers to about 40 micrometers, about 30 micrometers to about 100 micrometers, about 30 micrometers to about 200 micrometers, about 30 micrometers to about 500 micrometers, about 30 micrometers to about 1,000 micrometers, about 30 micrometers to about 2,000 micrometers, about 30 micrometers to about 3,000 micrometers, about 30 micrometers to about 5,000 micrometers, about 40 micrometers to about 100 micrometers, about 40 micrometers to about 200 micrometers, about 40 micrometers to about 500 micrometers, about 40 micrometers to about 1,000 micrometers, about 40 micrometers to about 2,000 micrometers, about 40 micrometers to about 3,000 micrometers, about 40 micrometers to about 5,000 micrometers, about 100 micrometers to about 200 micrometers, about 100 micrometers to about 500 micrometers, about 100 micrometers to about 1,000 micrometers, about 100 micrometers to about 2,000 micrometers, about 100 micrometers to about 3,000 micrometers, about 100 micrometers to about 5,000 micrometers, about 200 micrometers to about 500 micrometers, about 200 micrometers to about 1,000 micrometers, about 200 micrometers to about 2,000 micrometers, about 200 micrometers to about 3,000 micrometers, about 200 micrometers to about 5,000 micrometers, about 500 micrometers to about 1,000 micrometers, about 500 micrometers to about 2,000 micrometers, about 500 micrometers to about 3,000 micrometers, about 500 micrometers to about 5,000 micrometers, about 1,000 micrometers to about 2,000 micrometers, about 1,000 micrometers to about 3,000 micrometers, about 1,000 micrometers to about 5,000 micrometers, about 2,000 micrometers to about 3,000 micrometers, about 2,000 micrometers to about 5,000 micrometers, or about 3,000 micrometers to about 5,000 micrometers. In some embodiments, the method comprises harvesting the cell cultured fibers when the average width of the cell cultured fibers is from about 10 micrometers, about 20 micrometers, about 30 micrometers, about 40 micrometers, about 100 micrometers, about 200 micrometers, about 500 micrometers, about 1,000 micrometers, about 2,000 micrometers, about 3,000 micrometers, or about 5,000 micrometers. In some embodiments, the method comprises harvesting the cell cultured fibers when the average width of the cell cultured fibers is from at least about 10 micrometers, about 20 micrometers, about 30 micrometers, about 40 micrometers, about 100 micrometers, about 200 micrometers, about 500 micrometers, about 1,000 micrometers, about 2,000 micrometers, or about 3,000 micrometers. In some embodiments, the method comprises harvesting the cell cultured fibers when the average width of the cell cultured fibers is from at most about 20 micrometers, about 30 micrometers, about 40 micrometers, about 100 micrometers, about 200 micrometers, about 500 micrometers, about 1,000 micrometers, about 2,000 micrometers, about 3,000 micrometers, or about 5,000 micrometers.

In some embodiments, the method comprises culturing a single cell type. In some embodiments, the method comprises culturing a mixture of two or more cell types. Non-limiting example of the cell types can include muscle cells or muscle cell precursors, endothelial cells or endothelial cell precursors, adipose cells or adipose cell precursors, connective tissue cells of connective tissue cell precursors, or a combination thereof. Additional exemplary cell types can include embryonic stem cells, induced pluripotent stem cells, satellite cells, mesenchymal stem cells, or hematopoietic stem cells. In some embodiments, the cell cultured fibers are cultured in a heterologous extracellular matrix. In some embodiments, the cells are cultivated in plates comprising, consisting of or coated at least partially with a heterologous extracellular matrix. In some embodiments, the cells are cultivated in plates comprising, consisting of or coated at least partially with a biocompatible material like fibers or hydrogels. In some embodiments, plates comprising fiber or nanofibers are exemplary confinement materials possessing one or more advantageous properties including biocompatible, optically transparent adjustable fibers, compatible with 3D and 2D cell culture, mimicry of 3D topography, or any combination thereof. In some embodiments, the optically transparent fibers allow for live-cell imaging and real time quantification of cell mobility of 3D cell culture. In some embodiments, the optically transparent fibers allow for live-cell imaging and real time quantification of cell mobility of 3D or 2D cell culture.

In some embodiments, hydrogels are exemplary confinement materials possessing one or more advantageous properties including: non-adherent, biocompatible, extrudable, bioprintable, non-cellular, of suitable strength, and not soluble in aqueous conditions. In some embodiments, suitable hydrogels are natural polymers. In further embodiments, suitable hydrogels include those derived from surfactant polyols such as Pluronic F-127, collagen, hyaluronate, fibrin, alginate, agarose, chitosan, dextran, and derivatives or combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, agarose, alginate, gelatin, Matrigel™ (e.g., solubilized basement membrane matrix secreted by Engelbreth-Holm-Swarm (EHS) mouse sarcoma cells, Corning Life sciences), hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, and combinations thereof.

In some embodiments, the cells are cultivated in plates, flasks or dishes compatible with cell culture comprising, consisting of or coated at least partially with a biocompatible material like a heterologous extracellular matrix. In some embodiments, the cells are cultivated in plates comprising, consisting of or coated at least partially with a biocompatible material like a heterologous extracellular matrix comprising 5-15% gelatinous protein mixture (for example secreted by Engelbreth-Holm-Swarm mouse sarcoma cells, also referred to as Matrigel). In some embodiments, the cells are cultivated in plates comprising, consisting of or coated at least partially with a biocompatible material like a heterologous extracellular matrix comprising 5-15% Matrigel™ or laminin. In some embodiments, a composition as previously described comprising multiple types of cells, wherein the cells are cultivated in plates comprising, consisting of, or coated at least partially with heterologous extracellular matrix comprising a volume of Matrigel™ ranging from about 1% to about 25%. In some embodiments, a composition as previously described comprising multiple types of cells, wherein the cells are cultivated in plates comprising, consisting of, or coated at least partially with heterologous extracellular matrix comprising a volume of Matrigel™ ranging from about 5% to about 15%. In some embodiments, a composition as previously described comprising multiple types of cells, wherein the cells are cultivated in plates comprising, consisting of, or coated at least partially with heterologous extracellular matrix comprising a volume of Matrigel™ ranging from about 6% to about 14%. In some embodiments, a composition as previously described comprising multiple types of cells, is cultivated in plates comprising, consisting of, or coated at least partially with heterologous extracellular matrix comprising a volume of Matrigel™ ranging from about 1% to about 5%, about 1% to about 7%, about 1% to about 10%, about 1% to about 12%, about 1% to about 15%, about 1% to about 20%, about 1% to about 25%, about 5% to about 7%, about 5% to about 10%, about 5% to about 12%, about 5% to about 15%, about 5% to about 20%, about 5% to about 25%, about 7% to about 10%, about 7% to about 12%, about 7% to about 15%, about 7% to about 20%, about 7% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, a composition as previously described comprising multiple types of cells, wherein the cells are cultivated in plates comprising, consisting of, or coated at least partially with heterologous extracellular matrix comprising a volume of Matrigel™ ranging from about 1%, about 5%, about 7%, about 10%, about 12%, about 15%, about 20%, or about 25%. In some embodiments, a composition as previously described comprising multiple types of cells, wherein the cells are cultivated in plates comprising, consisting of, or coated at least partially with heterologous extracellular matrix comprising a volume of Matrigel™ ranging from at least about 1%, about 5%, about 7%, about 10%, about 12%, about 15%, or about 20%. In some embodiments, a composition as previously described comprising multiple types of cells, wherein the cells are cultivated in plates comprising, consisting of, or coated at least partially with heterologous extracellular matrix comprising a volume of Matrigel™ ranging from at most about 5%, about 7%, about 10%, about 12%, about 15%, about 20%, or about 25%. The biocompatible material of the plates dishes or flasks may comprise suitable hydrogels that include those derived from surfactant polyols such as Pluronic F-127, collagen, hyaluronate, fibrin, alginate, agarose, chitosan, dextran, and derivatives or combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, agarose, alginate, gelatin, Matrigel™ (e.g., solubilized basement membrane matrix secreted by Engelbreth-Holm-Swarm (EHS) mouse sarcoma cells, Corning Life sciences), hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, and combinations thereof.

Plates, dishes or flasks for culturing the cells and/or spheroids described herein may comprise one or more recesses. The plates, dishes, and/or flasks may comprise at least two recesses. Preferably, the at least two recesses have the shape of a hemisphere, a spherical cap, a semi ellipsoid, a cone, a truncated cone, a terraced cone, a pyramid, a truncated pyramid, a terraced pyramid, a torus, or an elliptic paraboloid, among other shapes. Especially with two recesses in the shape of a hemisphere, round cellular spheroids can be produced with the microfluidic according to the invention. With the at least two recesses having the shape of a spherical cap, the spherical cap has a polar angle a of 30° to 90°, preferably 40° to 90°, more preferably 50° to 90°, more preferably 60° to 90°, more preferably 70° to 90°, more preferably 80° to 90°, and more preferably 85° to 90°. The plates, dishes or flasks may comprise at least 3, 4, 5, 6, 7, 8, 9, 10, 11, 12, 24, 48, 96, or 384 recesses.

Another aspect the present invention relates to the production of cellular spheroids comprising different metabolic activity following treatment with recombinant growth factors. In some embodiments, spheroids are treated with non-human recombinant growth factors or growth factors of the species from which the cells are sourced. In some embodiments, spheroids are treated with recombinant growth factors selected from the group consisting of vascular endothelial growth factor (VEGF (A-F)), fibroblast growth factors (acidic and basic FGF 1-10), granulocyte-macrophage colony-stimulating factor (GM-CSF), insulin, insulin growth factor or insulin-like growth factor (IGF), insulin growth factor binding protein (IGFBP), placenta growth factor (PIGF), angiopoietin (Ang1 and Ang2), platelet-derived growth factor (PDGF), hepatocyte growth factor (HGF), transforming growth factor (TGF-α, TGF-β, isoforms 1-3), platelet-endothelial cell adhesion molecule-1 (PECAM-1), vascular endothelial cadherin (VE-cadherin), nitric oxide (NO), chemokine (C—X—C motif) ligand 10 (CXCL10) or IP-10, interleukin-8 (IL-8), hypoxia inducible factor (HIF), monocyte chemotactic protein-1 (MCP-1), vascular cell adhesion molecule (VCAM), ephrin ligands (including Ephrin-B2 and -B4). Transcription factors include, but are not limited to, HIF-1α, HIF-1β and HIF-2α, Ets-1, Hex, Vezf1, Hox, GATA, LKLF, COUP-TFII, Hox, MEF2, Braf, Prx-1, Prx-2, CRP2/SmLIM, and GATA family members, basic helix-loop-helix factors, or any combination thereof. In some embodiments, spheroids are treated with recombinant growth factors selected from the group consisting of fibroblast growth factor (FGF), hepatocyte growth factor (HGF), and insulin-like growth factor (IGF). In some embodiments, spheroids are treated with one of these recombinant growth factors selected from the group consisting of fibroblast growth factor (FGF), hepatocyte growth factor (HGF), and insulin-like growth factor (IGF). In some embodiments, spheroids are treated with any of these recombinant growth factors selected from the group consisting of fibroblast growth factor (FGF), hepatocyte growth factor (HGF), and insulin-like growth factor (IGF). In some embodiments, spheroids are treated with fibroblast growth factor (FGF). In some embodiments, spheroids are treated with hepatocyte growth factor (HGF). In some embodiments, spheroids are treated with insulin-like growth factor (IGF). The growth factor cultured with the spheroids or cells or cell precursors described herein can be derived from the same animal or species as the spheroids, cells, or cell precursors, or from a different animal or species as the spheroids, cells, or cell precursors. The growth factors can be recombinantly produced.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such levels. The decrease can be, for example, at least 10%, at least 20%, at least 30%, at least 40% or more, and is preferably down to a level accepted as within the range of normal for an individual without a given disease.

As used herein the term “non-human animal” refers to live organisms that are not human or not Homo sapiens species. In certain embodiments, the individual is a mammal. In certain embodiments, the mammal is a mouse, rat, rabbit, dog, cat, horse, cow, sheep, pig, goat, llama, alpaca, yak, bison, wagyu cattle, boar, elk, deer, or camel. In certain embodiments, the non-human animal is selected from the group consisting of: a cow, a pig, a chicken, a fish, a bird, a sheep, a bison, a wagyu cattle, a boar, a reptile, an ostrich, a sheep, a goat, a camel, a duck, a goose, an elk, a deer, and a turkey.

As used herein the term “non-human animal cells” refers to cells derived from a non-human animal. Examples of non-human animal cells are those cells that can differentiate into or that are derived from one or more types of tissues including muscle, connective tissue, fat, cartilage, liver, heart, eye, skin, lung, intestine, kidney tissue, bone marrow, umbilical cord, and embryonic tissue. The term “muscle cell” refers to a cells which contribute to skeletal contractile motion that form the skeletal muscle tissues of the body, which may include for example a myocyte. The term “muscle cell precursor” refers to myogenic stem cells such as satellite cells. Muscle cells and muscle cell precursors may be isolated from the body of an animal using for example a tissue biopsy. The term “fat cell” refers to a cell that has differentiated and become specialized in the synthesis and storage of fat. In certain embodiments, a fat cell is a lipocyte or adipocyte. In certain embodiments, a “fat cell precursor” refers to cells that develop into fat cells, such as mesenchymal stem cells. In certain embodiments, differentiated fat cells or precursor mesenchymal stem cells may be isolated from the body of an animal using for example a tissue biopsy. The term “connective tissue cell” refers to any of the cells that secrete or differentiate into cells that secrete extracellular matrix or that may develop into or are of the specialized connective tissue of the body, including but not limited to, areolar, dense, elastic, reticular blood, bone, cartilage, collagen, or any combination thereof. In certain embodiments, a connective tissue cell comprises the tissue that connects, separates, and supports all other types of tissues in the body. In certain embodiments, a connective tissue cell is a fibroblast. In certain embodiments, a fibroblast is a type of biological cell that synthesizes the extracellular matrix and collagen, and produces the structural framework (stroma) for animal tissues. In certain embodiments, a fibroblast or connective tissue cell may be derived from mesenchymal stem cells and/or mesenchyme. The term “cartilage cell” or “chondrocyte” refers to a cell that has differentiated and become specialized in the synthesis and turnover of a large volume of extracellular matrix (ECM) components such as collagen, glycoproteins, proteoglycans, and hyaluronan. In certain embodiments, chondrocytes vary according to positioning, such as, for example, articular cartilage, including the deep zone, epiphyseal plates, and tissue boundaries. In certain embodiments, a chondrocyte or cartilage cell may be derived from mesenchymal stem cells. The term “chondrocyte precursor” includes cells that develop into chondrocytes, for example, mesenchymal stem cells.

As used herein the term “marbled” refers to a pattern of intramuscular fat tissue within muscle tissue. In some embodiments, the pattern of intramuscular fat tissue contributes to the meat tenderness, juiciness, texture, flavor, appearance, or any combination thereof. In some embodiments the muscle tissue is lean muscle tissue.

As used herein the term “3D”, “3D formation” or “three-dimensional structure” refers to having three dimensions such as height, weight, and depth (or thickness).

As defined herein, a “spheroid” or “organoid” is a type of 3D cell modeling that can simulate a live cell's environmental conditions as compared to a 2D cell model, specifically with the reactions between cells and the reactions between cells and the matrix. Spheroids are useful in the study of changing physiological characteristics of cells, the difference in the structure of healthy cells and tumor cells, and the changes cells undergo when forming a tumor. Spheroids herein may be referred to as a type of spheroid. Spheroids of different types refer to distinct spheroids that are compositionally distinct. For example, a first type of spheroid may predominantly comprise muscle cells or muscle cell precursors, while a second type of spheroid may predominantly comprise adipose tissue or adipose tissue precursors. Spheroid types may comprise heterogenous mixtures of different cell types.

As defined herein, an “organoid” is a miniaturized and simplified version of an organ produced in vitro in 3D that shows realistic micro-anatomy. Organoids may be derived from one or a few cells from a tissue, stem cells, hematopoietic stem cells, mesenchymal stem cells, bone marrow cells, embryonic stem cells, induced pluripotent stem cells, precursor cells, or differentiated progenitor cells which can self-organize in three-dimensional culture owing to their self-renewal and differentiation capacities.

A “cellular spheroid”, as used herein, is a 3D cell aggregate in the form of a spheroid or having a spheroid-like form. Cellular spheroids can be formed by eukaryotic cells, and in particular, mammalian cells (e.g. human cells), whereby particularly preferred cells are cells being present in organs and tissues of mammals. These spheroids may comprise one or more type of cells. Spheroids function as a promising model for assessing therapeutic treatments, like chemotherapy, cell- and antibody based immunotherapy, gene therapy and combinatorial therapies. The 3D spheroid model can be used to improve the delivery system for compound penetration and targeting into tissues. The use of different type of cells allows to produce more complex “organoids” or tissue-like structures. Moreover, the spheroids can be loosely aggregated, and thus, represent a miniaturized model of a high density cell culture. Further, there is no requirement that spheroids need to stay in the 3D organization for scale up; in fact, they may dissociate.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

Embodiment 1. A method of acquiring a scaffold for a cultivated meat product comprising: forming hydrogel fibers; crosslinking the hydrogel fibers to obtain crosslinked hydrogel fibers; and lyophilizing the crosslinked hydrogel fibers to obtain crosslinked lyophilized hydrogel fibers.

Embodiment 2. The method of Embodiment 1, wherein the hydrogel fibers comprise: agarose, alginate, amino acid, cellulose, cellulose derivatives, chitosan, collagen, ethylene polyoxide, ethylene polyoxide copolymers, fibrin, gelatin, gelatin derivatives, hyaluronate, hyaluronan, hyaluronic acid methacrylate (HA-MA), hydroxyethyl methacrylate, lactic acid polymers, lipids, Matrigel™, natural polymers, Pluronic F-127, polyethylene glycol, polylactide-co-glycolide, polyacrylic acids, polyacrylic acids derivatives, polyvinyl alcohol, polyphosphazene, poloxamer, polysaccharides, proteins, peptides, poly-isopropyl-n-polyacrylamide, polyethylene glycol diacrylate (PEG-DA), polydimethylsiloxane, polyacrylamide, or any combination thereof.

Embodiment 3. The method of Embodiment 1, wherein the hydrogel fibers comprise: agarose, alginate, amino acid, cellulose, cellulose derivatives, chitosan, collagen, fibrin, gelatin, gelatin derivatives, hyaluronate, hyaluronan, hyaluronic acid methacrylate (HA-MA), lipids, polyethylene glycol, polylactide-co-glycolide, polysaccharides, proteins, crosslinking precursor component(s) or any combination thereof.

Embodiment 4. The method of Embodiment 1, wherein the hydrogel fibers comprise gelatin.

Embodiment 5. The method of any one of Embodiments 1 to 4, wherein the hydrogel fibers are formed from a concentration of about 1% to about 90% (w/v) hydrogel in an aqueous solution.

Embodiment 6. The method of any one of Embodiments 1 to 4, wherein the hydrogel fibers are formed from a concentration of about 5% to about 30% (w/v) hydrogel in an aqueous solution.

Embodiment 7. The method of any one of Embodiments 1 to 4, wherein the hydrogel fibers are formed from a concentration of about 5%, 6%, 7%, 8%, 9%, or 10% (w/v) hydrogel in an aqueous solution.

Embodiment 8. The method of any one of Embodiments 1 to 4, wherein the hydrogel fibers are formed from a concentration of about 8% (w/v) hydrogel in an aqueous solution.

Embodiment 9. The method of any one of Embodiments 1 to 8, wherein the hydrogel fibers are formed at about 4° C. to about 50° C.

Embodiment 10. The method of any one of Embodiments 1 to 8, wherein the hydrogel fibers are formed at about 37° C.

Embodiment 11. The method of any one of Embodiments 1 to 10, wherein forming the hydrogel fibers comprises filling a vacuum assembly fitted with a mesh with micro-sized apertures with liquid hydrogel, allowing the liquid hydrogel to cool and solidify to obtain solid hydrogel, and extruding the solid hydrogel through the fitted assembly mesh to obtain hydrogel fibers.

Embodiment 12. The method of Embodiment 11, wherein vacuum assembly chamber pressure is from about 5 millitorr to about 4000 millitorr.

Embodiment 13. The method of any one of Embodiments 1 to 12, wherein forming the hydrogel fibers comprises extrusion.

Embodiment 14. The method of any one of Embodiments 1 to 13, wherein crosslinking the hydrogel fibers to obtain crosslinked hydrogel fibers comprises: suspending the hydrogel fibers in an aqueous solution comprising at least one crosslinking reagent to obtain suspended hydrogel fibers; and maintaining the suspended hydrogel fibers for at least 6 hours at a temperature from about 0° C. to about 10° C. to obtain crosslinked hydrogel fibers.

Embodiment 15. The method of any one of Embodiments 1 to 14, wherein crosslinking the hydrogel fibers to obtain crosslinked hydrogel fibers comprises suspending the hydrogel fibers in an aqueous solution comprising at least one crosslinking reagent selected from a group consisting of: homobifunctional crosslinking reagents, heterobifunctional crosslinking reagents, photoreactive crosslinking reagents, disuccinimidyl suberate (DSS), disuccinimidyl tartrate (DST), dithiobis succinimidyl propionate (DSP), sulfhydryl-to-sulfhydryl crosslinkers, bismaleimidoethane (BMOE), dithiobismaleimidoethane (DTME), m-Maleimidobenzoyl-N-hydroxysuccinimide ester (MDS), N-γ-Maleimidobutyryloxysuccinimide ester (GMBS), N-(ε-Maleimidocaproyloxy) succinimide ester (EMCS), N-(ε-Maleimidocaproyloxy) sulfo succinimide ester (sulfo-EMCS), aryl azides, N-((2-pyridyldithio)ethyl)-4-azidosalicylamide, diazirines, genipin, N-hydroxysuccinimide (NHS) ester, 1-Ethyl-3-[3-dimethylaminopropyl]carbodiimide hydrochloride (EDC or EDAC), hexamethylenetetramine, glutaraldehyde, epoxy compounds, isocyanates, or any combination thereof.

Embodiment 16. The method of any one of Embodiments 1 to 15, wherein crosslinking the hydrogel fibers to obtain crosslinked hydrogel fibers comprises suspending the hydrogel fibers in an aqueous solution comprising at least one crosslinking agent selected from N-hydroxysuccinimide (NHS) ester, 1-Ethyl-3-[3-dimethylaminopropyl]carbodiimide hydrochloride (EDC or EDAC), genipin, or any combination thereof.

Embodiment 17. The method of any one of Embodiments 1 to 16, wherein crosslinking the hydrogel fibers to obtain crosslinked hydrogel fibers comprises shaker cultivating the suspended hydrogel fibers for 6 hours to 24 hours at a temperature from about 0° C. to about 10° C.

Embodiment 18. The method of any one of Embodiments 1 to 16, wherein crosslinking the hydrogel fibers to obtain crosslinked hydrogel fibers comprises shaker cultivating the suspended hydrogel fibers for no more than 12 hours at a temperature from about 0° C. to about 10° C.

Embodiment 19. The method of any one of Embodiments 1 to 18, wherein crosslinking the hydrogel fibers to obtain crosslinked hydrogel fibers comprises shaker cultivating the suspended hydrogel fibers from about 50 revolutions per minute to about 500 revolutions per minute.

Embodiment 20. The method of any one of Embodiments 1 to 10, wherein crosslinking the hydrogel fibers comprises a photo-crosslinking reaction.

Embodiment 21. The method of Embodiment 20, wherein the photo-crosslinking reaction comprises exposure to ultraviolet light.

Embodiment 22. The method of Embodiment 21, wherein the ultraviolet light comprises a wavelength of about 200 nanometers to about 600 nanometers.

Embodiment 23. The method of Embodiment 22, wherein the ultraviolet light comprises a wavelength of about 300 nanometers to about 400 nanometers.

Embodiment 24. The method of any one of Embodiments 1 to 23, wherein lyophilizing the crosslinked hydrogel fibers to obtain crosslinked lyophilized hydrogel fibers comprises: freezing the crosslinked hydrogel fibers at a first temperature sufficient to transform the water in crosslinked hydrogel fibers from liquid state to solid state; and drying the crosslinked hydrogel fibers at a second temperature sufficient to remove the water by sublimation from the crosslinked hydrogel fibers.

Embodiment 25. The method of Embodiment 24, further comprises washing the crosslinked hydrogel fibers.

Embodiment 26. The method of Embodiment 24, wherein the first temperature is from about −100° C. to about −10° C.

Embodiment 27. The method of Embodiment 24, wherein the second temperature is from about −50° C. to about 50° C.

Embodiment 28. The method of Embodiment 24, wherein the crosslinked hydrogel fibers are dried in a vacuum chamber at pressure from about 5 millitorr to about 4000 millitorr.

Embodiment 29. The method of any one of Embodiments 1 to 28, wherein the lyophilized crosslinked hydrogel fibers are sterilized to obtain sterilized lyophilized crosslinked hydrogel fibers, wherein sterilization comprises: heating or irradiating the lyophilized crosslinked hydrogel fibers; and/or immersing the lyophilized crosslinked hydrogel fibers in alcohol-based soaking solution.

Embodiment 30. The method of Embodiment 29, wherein the heating comprises heating the lyophilized crosslinked hydrogel fibers with a temperate between about 37° C. to about 121° C.

Embodiment 31. The method of Embodiment 29, wherein the irradiating comprises contacting the lyophilized crosslinked hydrogel fibers with UV radiation.

Embodiment 32. The method of any one of Embodiments 1 to 31, wherein the hydrogel fibers are characterized by a flexible dissolution rate from about 3 minutes to more than 100 days.

Embodiment 33. The method of any one of Embodiments 1 to 31, wherein the hydrogel fibers are characterized by controllable gelation time from about 5 seconds to about 12 minutes.

Embodiment 34. The method of any one of Embodiments 1 to 33, wherein the hydrogel fibers possess an average width from about 40 micrometers to about 1000 micrometers.

Embodiment 35. The method of any one of Embodiments 1 to 33, wherein the hydrogel fibers possess an average width from about 40 micrometers to about 250 micrometers.

Embodiment 36. The method of any one of Embodiments 1 to 35, wherein the hydrogel fibers possess an average length of from about 150 micrometers to about 12 centimeters.

Embodiment 37. The method of any one of Embodiments 1 to 35, wherein the hydrogel fibers possess an average density from about 19 mole per cubic meter to about 56 mole per cubic meter.

Embodiment 38. The method of any one of Embodiments 1 to 37, wherein the hydrogel fibers comprise low-rigidity elasticity from about 2 kilopascals to about 30 kilopascals.

Embodiment 39. The method of any one of Embodiments 1 to 38, wherein the hydrogel fibers are water-stable.

Embodiment 40. The method of any one of Embodiments 1 to 39, wherein the hydrogel fibers comprise a porous surface wherein an average pore size opening possesses a width from about 2 micrometers to about 500 micrometers.

Embodiment 41. The method of any one of Embodiments 1 to 40, wherein the hydrogel fibers are bio-compatible.

Embodiment 42. The method of any one of Embodiments 1 to 41, wherein the hydrogel fibers comprise a thermoreversible hydrogel that is not a liquid at room temperature.

Embodiment 43. The method of any one of Embodiments 1 to 42, wherein the hydrogel fibers comprise a thermoreversible hydrogel with a gelation temperature (Tgel) from about 10° C. to about 40° C.

Embodiment 44. The method of any one of Embodiments 1 to 43, further comprising contacting the scaffold with cells or cell precursors from a non-human animal source.

Embodiment 45. The method of Embodiment 44, wherein the cells or cell precursors from a non-human animal source comprise cells from a tissue biopsy, an immortalized cell line, blood, stem cells, precursor cells, embryonic cells, bone marrow, or any combination thereof.

Embodiment 46. The method of Embodiment 44 or 45, wherein the method includes screening cells or cultured fibers for metabolic activity.

Embodiment 47. The method of Embodiment 44 or 45, wherein the method includes expanding the cells or cell precursors for 12 hours to 10 days to obtain cell cultured fibers.

Embodiment 48. The method of any one of Embodiments 1 to 47, comprising harvesting the cell cultured fibers when the average width of the cell cultured fibers is from about 40 micrometers to about 2000 micrometers.

Embodiment 49. The method of any one of Embodiments 44 to 48, wherein the cells comprise a single cell type.

Embodiment 50. The method of any one of Embodiments 44 to 49, wherein the cells comprise a mixture of two or more cell types.

Embodiment 51. The method of Embodiment 49 or 50, wherein the single cell type or the two or more cell types are selected from muscle cells or muscle cell precursors, endothelial cells or endothelial cell precursors, adipose cells or adipose cell precursors, connective tissue cells of connective tissue cell precursors, or a combination thereof.

Embodiment 52. The method of any one of Embodiments 49 to 51, wherein the cell cultured fibers further comprise embryonic stem cells, induced pluripotent stem cells, satellite cells, mesenchymal stem cells, and/or hematopoietic stem cells.

Embodiment 53. The method of any one of Embodiments 44 to 52, wherein the non-human animal is selected from the group consisting of: a cow, a pig, a chicken, a fish, a sheep, a bison, a duck, a goose, an elk, a deer, a Berkshire pig, a Kurobuta pig, an Iberian pig, an ostrich, and combinations thereof.

Embodiment 54. The method of any one of Embodiments 47 to 53, wherein the cell cultured fibers are cultured in a heterologous extracellular matrix.

### EXAMPLES

The following illustrative examples are representative of embodiments of the stimulation, systems, and methods described herein and are not meant to be limiting in any way.

### Example 1. Methods for Making, Lyophilizing, or Growing Cells on Hydrogel Fibers

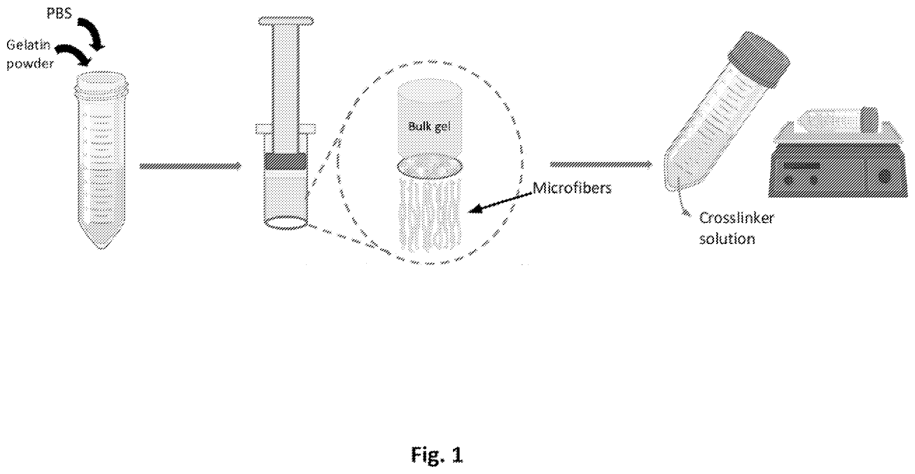

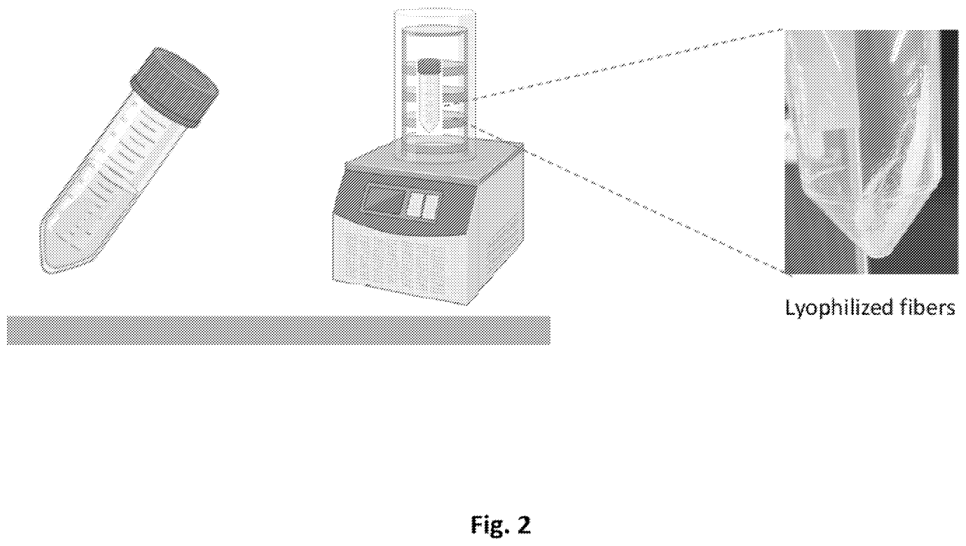

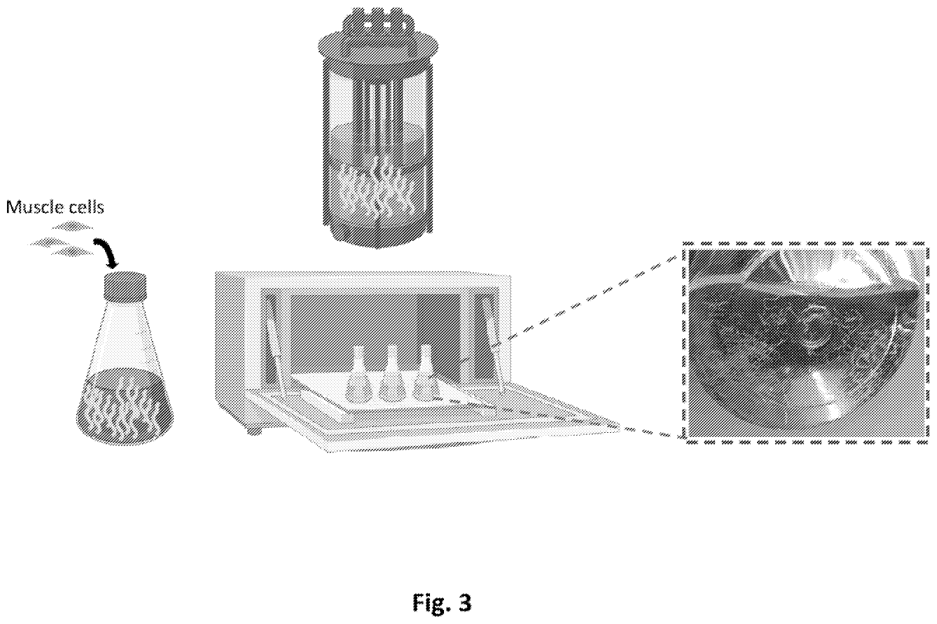

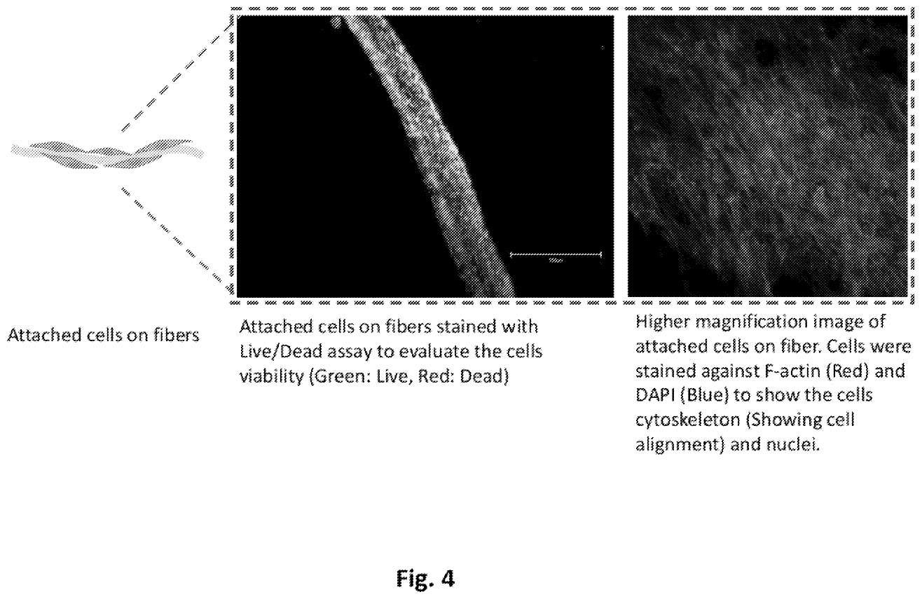

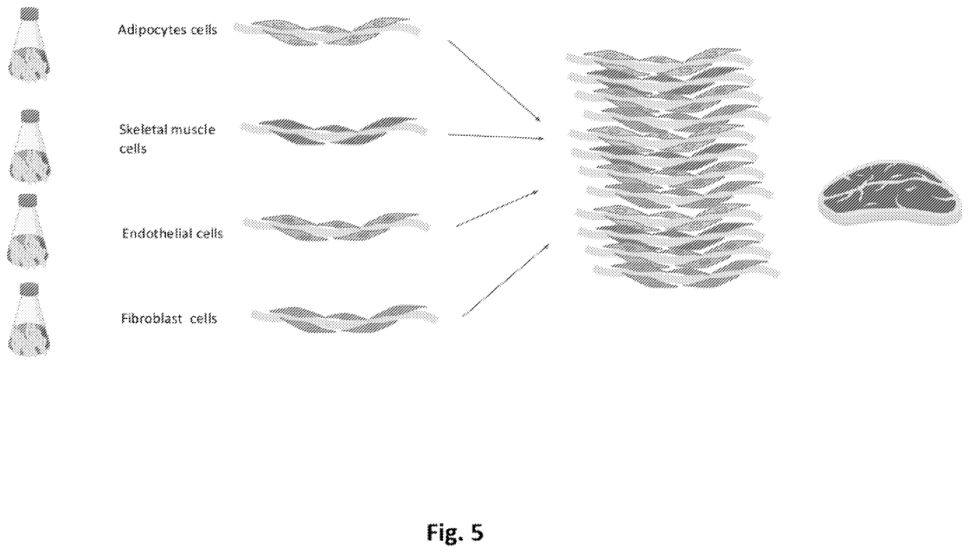

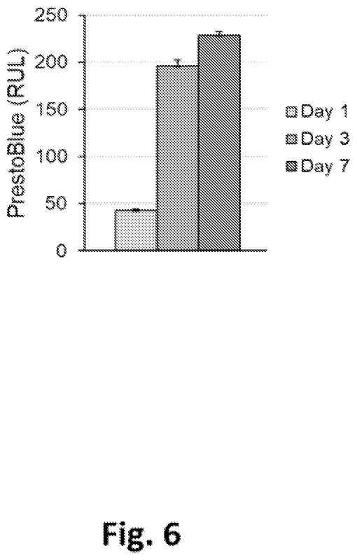

As shown in FIG. 1, one method of making the fibers described herein comprises mixing 5 to 20% gelatin with a buffered solution such as PBS at 37° C. to form the hydrogel followed by extrusion of the hydrogel through a mesh with a 40 to 200 micrometer defined pore size, the hydrogel fibers can then be crosslinked using EDC (1-ethyl-3-(−3-dimethylaminopropyl) carbodiimide hydrochloride)/NHS (N-hydroxysuccinimide) chemistry with shaking overnight at 4° C. As shown in FIG. 2 the fibers can be washed in deionized water/ethanol solution to washout the residue of unreacted crosslinker, then washed with DI water, and lyophilized using a standard lyophilization/freeze-drying apparatus, to yield lyophilized hydrogel fibers (FIG. 2, far right). As shown in FIG. 3 fibers could be seeded with cells (6 million or fewer cells to 50 mg of freeze dried fibers, in this example, muscle cells) in a tissue culture flask and incubated in an orbital shaker (for 3 to 7 days) at 37° C. and 5% CO₂ to yield cells grown on fibers (FIG. 3, far right). FIG. 4 shows that cells associated with the fibers were alive and displayed normal morphology. FIG. 5 shows that this process can be repeated with other cell types and those cell-fiber compositions can be admixed to replicate the complexities of muscle tissue and meat. FIG. 6 shows an exemplary graph of cell fiber proliferation across seven days cultured by the method described in this example.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method of acquiring a scaffold for a cultivated meat product comprising:
a) forming hydrogel fibers;
b) crosslinking the hydrogel fibers to obtain crosslinked hydrogel fibers; and
c) lyophilizing the crosslinked hydrogel fibers to obtain crosslinked lyophilized hydrogel fibers.

2-60. (canceled)

61. The method of claim 1 further comprising culturing cells on the crosslinked lyophilized hydrogel fibers to obtain cell cultured crosslinked lyophilized hydrogel.

62. The method of claim 1, wherein forming the hydrogel fibers comprises filling an assembly fitted with a mesh with micro-sized apertures with liquid hydrogel, allowing the liquid hydrogel to cool and solidify to obtain solid hydrogel, and extruding the solid hydrogel through the fitted assembly mesh to obtain hydrogel fibers.

63. The method of claim 62, wherein the assembly comprises an extruder.

64. The method of claim 62, wherein the assembly comprises an assembly chamber pressure.

65. The method of claim 1, wherein crosslinking the hydrogel fibers to obtain crosslinked hydrogel fibers comprises:
suspending the hydrogel fibers in an aqueous solution comprising at least one crosslinking reagent to obtain suspended hydrogel fibers; and
maintaining the suspended hydrogel fibers for at least 6 hours at a temperature from about 0° C. to about 10° C. to obtain crosslinked hydrogel fibers.

66. The method of claim 1, wherein lyophilizing the crosslinked hydrogel fibers to obtain crosslinked lyophilized hydrogel fibers comprises:
freezing the crosslinked hydrogel fibers at a first temperature sufficient to transform the water in crosslinked hydrogel fibers from liquid state to solid state; and
drying the crosslinked hydrogel fibers at a second temperature sufficient to remove the water by sublimation from the crosslinked hydrogel fibers.

67. The method of claim 66, further comprises washing the crosslinked hydrogel fibers.

68. The method of claim 1, wherein the lyophilized crosslinked hydrogel fibers are sterilized to obtain sterilized lyophilized crosslinked hydrogel fibers, wherein sterilization comprises:
heating or irradiating the lyophilized crosslinked hydrogel fibers; and
immersing the lyophilized crosslinked hydrogel fibers in alcohol-based soaking solution.

69. The method of claim 1, wherein the hydrogel fibers comprise a porous surface wherein an average pore size opening possesses a width from about 2 micrometers to about 500 micrometers.

70. The method of claim 1, wherein the hydrogel fibers comprise a thermoreversible hydrogel that is not a liquid at room temperature.

71. The method of claim 1, wherein the hydrogel fibers are characterized by a flexible dissolution rate from about 3 minutes to more than 100 days.

72. The method of claim 1, wherein the hydrogel fibers comprise low-rigidity elasticity from about 2 kilopascals to about 30 kilopascals.

73. The method of claim 1, further comprising contacting the scaffold with cells or cell precursors from a non-human animal source.

74. The method of claim 73, wherein the non-human animal is selected from the group consisting of: a cow, a pig, a chicken, a fish, a sheep, a bison, a duck, a goose, an elk, a deer, a Berkshire pig, a Kurobuta pig, an Iberian pig, an ostrich, and combinations thereof.

75. The method of claim 73, wherein the method includes expanding the cells or cell precursors for 12 hours to 10 days to obtain cell cultured fibers.

76. The method of claim 75, comprising harvesting the cell cultured fibers when the average width of the cell cultured fibers is from about 40 micrometers to about 2000 micrometers.

77. The method of claim 75, wherein the cell cultured fibers are cultured in a heterologous extracellular matrix.

78. The method of claim 73, wherein the cells or cell precursors comprise cells or cell precursors from a mixture of two or more cell types.

79. The method of claim 78, wherein the two or more cell types are selected from muscle cells or muscle cell precursors, endothelial cells or endothelial cell precursors, adipose cells or adipose cell precursors, connective tissue cells of connective tissue cell precursors, or a combination thereof.
